(19) United States
(12) Patent Application Publication
(10) Pub. No.: US 2025/0043005 A1
(43) Pub. Date: Feb. 6, 2025

(54) ANTIBODY-CYTOKINE FUSION PROTEINS AND APPLICATIONS THEREOF

(71) Applicant: GENEQUANTUM HEALTHCARE (SUZHOU) CO., LTD., Suzhou (CN)

(72) Inventors: Gang QIN, Suzhou (CN); Paul H. SONG, Suzhou (CN); Dong YANG, Suzhou (CN); Cao LV, Suzhou (CN)

(21) Appl. No.: 18/717,575

(22) PCT Filed: Dec. 8, 2022
(86) PCT No.: PCT/CN2022/137449

§ 371 Date: Jun. 7, 2024

(30) Foreign Application Priority Data
Dec. 9, 2021 (WO) PCT/CN2021/136727

(51) Int. Cl. C07K 16/28; A61K 39/00; A61P 35/00; C07K 14/54

(52) U.S. Cl. CPC C07K 16/2827; A61P 35/00; C07K 14/54; A61K 2039/505; C07K 2317/73; C07K 2317/92; C07K 2319/50

(57) ABSTRACT

Provided are antibody-cytokine fusion proteins comprising an anti-PD-L1 antibody and an Interleukin 21, and methods for preparing the same, as well as applications of the fusion proteins for therapeutic purpose.

### CROSS REFERENCE TO RELATED APPLICATIONS

This application is a National Stage Entry under 35 U.S.C. § 371 of International Application No. PCT/CN2022/137449 filed Dec. 8, 2022, which claims priority to International Application No. PCT/CN2021/136727 filed Dec. 9, 2021, the contents of each of which are hereby incorporated by reference in their entireties.

### REFERENCE TO A SEQUENCE LISTING

This application contains a Sequence Listing as a separate part of the disclosure. The contents of the Sequence Listing (2024-06-10-GQH-17-US-Sequence Listing.xml; Size: 60,497 bytes; and Date of Creation: Jun. 10, 2024) are incorporated herein by reference.

### TECHNICAL FIELD

The present disclosure relates to the field of biopharmaceuticals. In particular, provided are antibody-cytokine fusion proteins comprising an anti-PD-L1 antibody and an Interleukin 21, methods for preparing the same and applications thereof.

### BACKGROUND

Interleukin 21 (IL21) is a 4α-helix bundle cytokine secreted primarily by CD4⁺ T cells, B cells, myeloid cells and natural killer (NK) T cells. Signaling through heterodimers of the IL21 receptor (IL21R) and the common cytokine receptor gamma chain, IL21 exerts pleiotropic effects on a broad range of immune cell types and regulates both innate and adaptive immune responses. It has been reported that intratumoral injection of recombinant IL21 could promote the shift of tumor-associated macrophages within the tumor microenvironment from an immune-suppressive M2 phenotype to a tumor-inhibiting M1 phenotype. Anti-tumor activity of IL21 has been demonstrated in phage I or phage II clinical trials of renal cell carcinoma, Non-Hodgkin's lymphoma and melanoma, either as monotherapy or in combination with other therapeutics. However, IL21 has a very short half-life and causes significant side effects when systemically administered. Intratumoral administration of recombinant IL21 offers possible advantages including higher efficacy and lower systematic toxicity but is rather difficult for most patients.

Programmed cell death protein 1 (PD-1) and programmed cell death ligand 1 (PD-L1) are immune inhibitory receptor/ligand regulating T cell proliferation and activity. The PD-1/PD-L1 pathway is exploited by cancer cells to suppress the anti-tumor immunity and is generally associated with poor clinical outcomes. PD-1/PD-L1 blockade using antibodies against PD-1 or PD-L1 has shown promising therapeutic effects in the treatment of a wide variety of cancer types. Despite the encouraging clinical efficacy, only a minority of patients benefit from anti-PD-1/PD-L1 immunotherapies.

### SUMMARY

In one aspect, provided is a fusion protein having the structure of formula (I) or (I′):

[B-Sp₁-Lk-Sp₂]ₘ-A (I)

A-[Sp₁-Lk-Sp₂-B]ₘ (I′)
wherein
A is an anti-PD-L1 antibody;
B is an interleukin 21 (IL21);
Sp₁ and Sp2 are each independently a spacer or absent,
Lk is a linker formed by conjugation of an acceptor substrate recognition sequence and a donor substrate recognition sequence of a Sortase;
m is an integer of 1-4.

In some embodiments, the acceptor substrate recognition sequence comprises 1-100 series-connected structure units which are selected from the group consisting of glycine (G) and alanine (A). In a particular embodiment, the acceptor substrate recognition sequence comprises the sequence of (G)ₙ, wherein n is an integer of 2-20, especially 3.

In some embodiments, the donor substrate recognition sequence comprises the sequence of X₁X₂X₃TX₄X₅, wherein, X₁ is leucine (L) or asparagine (N), X₂ is proline (P) or alanine (A), X₃ is any natural or unnatural amino acid, X₄ is glycine (G), serine (S) or asparagine (N) or absent, X₅ is an amino acid sequence comprising 1-10 amino acids or absent.

In a particular embodiment, X₁X₂X₃TX₄X₅ is LPX₃TG, LPX₃TGG, NPQTN, NPKTG, LAX₃TG or LPQTSEQ, wherein X₃ is any natural or unnatural amino acid. In a preferred embodiment, X₁X₂X₃TX₄X₅ is LPETG or LPETGG.

In some embodiments, the spacer comprises 1-100 series-connected structure units which are selected from the group consisting of glycine (G), alanine (A) and serine (S).

In some embodiments, the anti-PD-L1 antibody is Atezolizumab, Avelumab, Durvalumab, 3F2 or an engineered antibody therefrom. In a preferred embodiment, the anti-PD-L1 antibody comprises a light chain having the amino acid sequence of SEQ ID NO:1 and a heavy chain having the amino acid sequence of SEQ ID NO:2, or a light chain having the amino acid sequence of SEQ ID NO:3 and a heavy chain having the amino acid sequence of SEQ ID NO:4.

In some embodiments, the IL21 is a wild-type IL21 or a variant thereof. In a preferred embodiment, the IL21 comprises the amino acid sequence of SEQ ID NO:9 or SEQ ID NO:10.

In some embodiments, the IL21 is conjugated at the N-terminus of the light chain and/or heavy chain of the anti-PD-L1 antibody. In a preferred embodiment, the IL21 is conjugated at the N-terminus of the heavy chain of the anti-PD-L1 antibody.

In some embodiments, Lk is X₁X₂X₃T(G)ₙ, the fusion protein has the structure of (II) or (II′):

[B-Sp₁-X₁X₂X₃T(G)ₙ-Sp₂]ₘ-A (II)

A-[Sp₁-X₁X₂X₃T(G)ₙ-Sp₂-B]ₘ (II′)
wherein, X₁ is leucine (L) or asparagine (N), X₂ is proline (P) or alanine (A), X₃ is any natural or unnatural amino acid, n is an integer of 2-20,
m is 1 or 2;
A, B, Sp₁ and Sp₂ are as defined above.

In a particular embodiment, the fusion protein has the structure of (III) or (III′):

[B-GALPETGGGGSGGGGS]ₘ-A (III)

A-[GALPETGGGGSGGGGS-B]ₘ (III′)
wherein A and B are as defined above.

In a particular embodiment, the fusion protein has the structure of formula (III):

[B-GALPETGGGGSGGGGS]ₘ-A (III)
wherein
A is an anti-PD-L1 antibody comprising a light chain having the amino acid sequence of SEQ ID NO:1 and a heavy chain having the amino acid sequence of SEQ ID NO:2, or a light chain having the amino acid sequence of SEQ ID NO:3 and a heavy chain having the amino acid sequence of SEQ ID NO:4;
B is an IL21 comprising the amino acid sequence of SEQ ID NO:9 or SEQ ID NO:10;
m is 1 or 2.

In a particular embodiment, the fusion protein comprises two identical heavy chain moieties and two identical light chain moieties, wherein
(1) each of the heavy chain moieties comprises the amino acid sequence of SEQ ID NO:18 or SEQ ID NO:20; and each of the light chain moieties comprises the amino acid sequence of SEQ ID NO:1; or
(2) each of the heavy chain moieties comprises the amino acid sequence of SEQ ID NO:19 or SEQ ID NO:21; and each of the light chain moieties comprises the amino acid sequence of SEQ ID NO:3.

In another particular embodiment, the fusion protein comprises two different heavy chain moieties and two identical light chain moieties, wherein
(1) one of the heavy chain moieties comprises the amino acid sequence of SEQ ID NO:18 or SEQ ID NO:20, the other one of the heavy chain moieties comprises the amino acid sequence of SEQ ID NO:14; and each of the light chain moieties comprises the amino acid sequence of SEQ ID NO:1; or
(2) one of the heavy chain moieties comprises the amino acid sequence of SEQ ID NO:19 or SEQ ID NO:21, the other one of the heavy chain moieties comprises the amino acid sequence of SEQ ID NO:15; and each of the light chain moieties comprises the amino acid sequence of SEQ ID NO:3.

In another aspect, provided is use of the fusion protein according to the present disclosure in the manufacture of a medicament for treating a disease in a subject in need thereof. Preferably, the disease is a cancer. More preferably, the cancer is selected from melanoma, ovarian cancer, breast cancer, Merkel cell carcinoma, lung cancer, renal cell cancer, bladder cancer, colon cancer, colon adenocarcinoma, colorectal cancer, head and neck squamous cell carcinoma, mesothelioma, virally induced cancers, cervical cancer, nasopharyngeal cancer, soft tissue sarcomas, hematological malignancies, gastric cancer, gastric or gastroesophageal junction (GEJ) adenocarcinoma, esophagus cancer, squamous cell carcinoma of the esophagus, endometrial carcinoma, primary mediastinal B-cell lymphoma (PMBCL), urothelial carcinoma, microsatellite instability-high (MSI-H) or a mismatch repair deficient (dMMR) solid tumor, hepatocellular carcinoma, lymphoma, Hodgkin lymphoma, non-Hodgkin's lymphoma, diffuse large B-cell lymphoma, renal cell carcinoma (RCC), cutaneous squamous cell carcinoma (cSCC), triple-negative breast cancer (TNBC), non-small cell lung cancer and bladder cancer.

In a further aspect, provided is a method for producing the fusion protein according to the present disclosure, comprising the following steps:
(a-1) providing an antibody moiety having the structure of formula (I-1) and a cytokine moiety having the structure of formula (I-2)

[Lkₐ-Sp₂]ₘ-A (I-1)

B-Sp₁-Lk_{d} (I-2);
and
(b-1) conjugating the antibody moiety with the cytokine moiety in the presence of a Sortase to obtain the fusion protein having the structure of formula (I)

[B-Sp₁-Lk-Sp₂]ₘ-A (I);
or
(a-2) providing an antibody moiety having the structure of formula (I′-1) and a cytokine moiety having the structure of formula (I′-2)

A-[Sp₁-Lk_{d}]ₘ (I′-1)

Lkₐ-Sp₂-B (I′-2);
and
(b-2) conjugating the antibody moiety with the cytokine moiety in the presence of a Sortase to obtain the fusion protein having the structure of formula (I′)

A-[Sp₁-Lk-Sp₂-B]ₘ (I′);
wherein
Lkₐ is an acceptor substrate recognition sequence of the Sortase as defined above, Lk_{d} is a donor substrate recognition sequence of the Sortase as defined above;
A, B, Sp₁, Sp₂, Lk and m are as defined above.

### BRIEF DESCRIPTION OF THE DRAWINGS

FIG. 1 shows schematics diagrams of the fusion proteins according to some embodiments of the present disclosure.

FIGS. 2A and 2B shows the binding activity of Ab0030 and Ab0033 to hPD-L1 analyzed by ELISA, respectively.

FIG. 3 shows SDS-PAGE analysis of antibody-cytokine conjugation mixtures containing the fusion proteins prepared from Ab0030 and Ab0033.

FIG. 4 shows SDS-PAGE analysis of purified fusion proteins prepared from Ab0030 and Ab0033.

FIG. 5 shows HIC-HPLC profiles of the fusion proteins prepared from Ab0030 and Ab0033.

FIG. 6 shows HIC-HPLC profiles of the fusion proteins prepared from Ab0030 and Ab0033.

FIG. 7 shows the binding activity of the antibody alone or the fusion protein to hPD-L1 analyzed by ELISA.

FIG. 8 shows the binding activity of the antibody alone or the fusion protein to IL21R analyzed by ELISA.

FIG. 9 shows flow cytometry analysis of STAT3 phosphorylation (pSTAT3) signals in PBMCs stimulated with control (open histogram) or hIL21, Ab0033 and LC0033-1 (filled histograms).

FIG. 10 shows body weights of the experimental animals from the first efficacy study at indicated time points.

FIG. 11 shows tumor volumes of the experimental animals from the first efficacy study at indicated time points.

FIG. 12 shows body weights of the experimental animals from the second efficacy study at indicated time points.

FIG. 13 shows tumor volumes of the experimental animals from the second efficacy study at indicated time points.

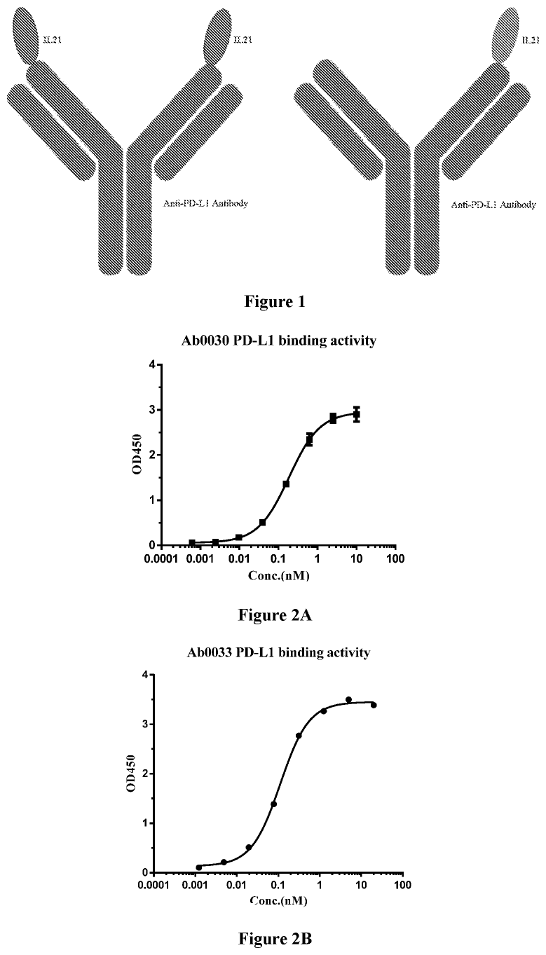

### DETAILED DESCRIPTION

### Definitions

Unless defined otherwise, all technical and scientific terms used herein have the same meaning as commonly understood by one of skill in the art. In addition, the terms and experimental procedures relating to biochemistry, molecular biology, cell and tissue culture, microbiology and immunology are those widely used in the art. Meanwhile, for better understanding of the present disclosure, definitions and explanations of relevant terms are provided below.

As used herein, the expression “at least one” or “one or more” means 1, 2, 3, 4, 5, 6, 7, 8, 9, 10 or more. Unless clearly indicated to the contrary, “a” and “an” as used herein should be understood as “at least one”.

When a certain amount, concentration, value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether said range is explicitly recited. Unless otherwise stated, the numerical ranges listed herein are intended to include the endpoints of the range and all integers and fractions (decimals) within the range. For example, the expression “i is an integer of 1 to 20” means that i is any integer of 1 to 20, for example, i can be 1, 2, 3, 4, 5, 6, 7, 8, 9, 10, 11, 12, 13, 14, 15, 16, 17, 18, 19 or 20. Other similar expressions such as n and m should also be understood in a similar manner.

As used herein, the term “optional” or “optionally” means the event described subsequent thereto may, but not necessarily happen, and the description includes the cases wherein said event or circumstance happens or does not happen.

The expressions “comprising”, “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, or groups of elements, steps or ingredients that are optionally present that do not substantially affect the essential and novel characteristics of the claimed subject matter. It should be understood that the expression “comprising” encompasses the expressions “consisting essentially of” and “consisting of”.

As used herein, the term “antibody” is an immunoglobulin (Ig) molecule or a fragment thereof that specifically binds to the epitope of an antigen through at least one antigen-binding site (i.e., the paratope). As used herein, the definition of “antibody (Ab)” encompasses conventional antibodies, recombinant antibodies, multispecific antibodies (e.g., bispecific antibodies), fully human antibodies, non-human antibodies, humanized antibodies, chimeric antibodies, intrabodies, diabodies, anti-idiotypic antibodies and antigen-binding fragments. The antigen-binding fragment as described herein can be produced by enzymatic treatment of full-length antibodies and/or synthetic methods, such as recombinant antibodies produced by grafting the CDRs of an antibody into the framework of another antibody. Examples of antigen-binding fragments include, but are not limited to, Fv, scFv, dsFv, scdsFv, diabody, Fab, scFab, Fab′, F(ab′)₂, and other fragments. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b). In some embodiments, the antibody is an anti-PD-L1 antibody which recognizes at least a portion of PD-L1. In some embodiments, the anti-PD-L1 antibody is a conventional antibody which comprises two identical light chains and two identical heavy chains.

A “conventional” or “full-length” antibody typically consists of four polypeptides: two heavy chains (HC) and two light chains (LC). Each light chain, from the amino-terminus to the carboxyl-terminus, comprises a light chain variable region (VL) and a light chain constant region (CL). Each heavy chain, from the amino-terminus to the carboxyl-terminus, comprises a heavy chain variable region (VH) and one or more heavy chain constant regions (CH).

Each VL and VH typically has three hypervariable “complementarity-determining regions (CDRs)” and four relatively conserved “framework regions (FRs)”, arranged from the amino-terminus to the carboxyl-terminus in the following order: FR1, CDR1, FR2, CDR2, FR3, CDR3 and FR4. According to the locations in the VL or VH, the CDR is designated as the heavy chain variable region CDR (CDRH), like CDRH1, CDRH2 and CDRH3; or the light chain variable region CDR (CDRL), like CDRL1, CDRL2 and CDRL3.

One of skill in the art knows and can define the CDRs using methodology well-known in the art, for example, using different numbering schemes, including Kabat (see e.g., Kabat, E. A. et al. (1991) Sequences of Proteins of Immunological Interest, Fifth Edition, U.S. Department of Health and Human Services, NIH Publication No. 91-3242), Chothia (see e.g., Chothia, C. et al. (1987) J. Mol. Biol. 196:901-917), AbM (see e.g., Martin, A. C. R. and Allen, J. (2007). Bioinformatics Tools for Antibody Engineering. In Handbook of Therapeutic Antibodies, S. Dübel (Ed.).) and IMGT (see e.g., Lefranc, M.-P., 2011 (6), IMGT, the International ImMunoGeneTics Information System Cold Spring Harb Protoc.; and Lefranc. M.-P. et al., Dev. Comp. Immunol., 27, 55-77 (2003)). Those skilled in the art would understand that the CDR sequences of a given antibody may have different boundaries depending on the numbering scheme used. Although the CDR sequences of the anti-PD-L1 antibody as described herein are defined using the Kabat numbering scheme, CDR sequences defined by any other numbering schemes shall fall into the protection scope of the present disclosure.

As used herein, the term “binding affinity” is a measure of the strength between two binding partners, such as an antibody and its cognate antigen (for example, anti-PD-L1 antibody and PD-L1) or a ligand and its receptor (for example, IL21 and IL21R). Typically, the affinity between proteins is usually measured and reported by the equilibrium dissociation constant K_{D}. K_{D} can be calculated from kinetic analysis using the following equation:

K_{D}=k_{off}/kₒₙ

wherein k_{off} and kₒₙ are the dissociation and association rate constants between the antigen and the antibody, respectively. The K_{D} and affinity are inversely related. Typically, the K_{D} between an antibody and its cognate antigen is about 10⁻⁶ M to about 10⁻⁹ M or less, such as 10⁻⁸ or less, 10⁻⁹ or less, or 10⁻¹⁰ or less. The k_{off} and kₒₙ can be measured using standard kinetic analysis methods, including, but not limited to, immunoassays, surface plasmon resonance (SPR) (Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, W. E., Fundamental Immunology, 2^{nd} ed., Raven Press, New York, pages 332-336 (1989); see also U.S. Pat. No. 7,229,619). Instrumentation and methods for real time detection and monitoring of binding rates are known and are commercially available (e.g., BiaCore 2000, Biacore AB, Upsala, Sweden).

As used herein, the term “polypeptide” refers to two or more amino acids covalently jointed by peptide bonds. The terms “polypeptide” and “protein” are used interchangeably herein. Polypeptides can be modified to include, for example, one or more spacers or functional moieties (for example, polyethylene glycols (PEGylation) and glycans (glycosylation)) and the like. Methods for modifying polypeptides are well known to those skilled in the art.

As used herein, a “natural amino acid” refers to a naturally occurring amino acid in the L-configuration used for protein synthesis in living organisms. Natural amino acids are represented herein by the standard one-letter or three-letter codes as known to those skilled in the art: Alanine (A; Ala), Arginine (R; Arg), Asparagine (N; Asn), Aspartic acid (D; Asp), Cysteine (C; Cys), Glutamine (Q; Gln), Glutamic acid (E; Glu), Glycine (G; Gly), Histidine (H; His), Isoleucine (I; Ile), Leucine (L; Leu), Lysine (K; Lys), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), Pyrrolysine (O; Pyl), Selenocysteine (U; Sec). Examples of unnatural amino acids include D-isomers and derivatives of the natural amino acids as described above and unusual amino acids. Unnatural amino acids include but are not limited to Homo-amino acids, Beta-homo-amino acids, N-methyl amino acids, Alpha-methyl amino acids, Citrulline (Cit), Hydroxyproline (Hyp), Norleucine (Nle), 3-nitrotyrosine, Nitroarginine, Ornithine (Orn), Naphtylalanine (Nal), Aminobutyric Acid (Abu), Diaminobutyric Acid (Dab), Methionine sulfoxide and Methionine sulfone.

As used herein, the numbering of amino acid positions in a polypeptide is defined as follows: the position of the first amino acid from the N-terminus is designated as position 1.

As used herein, a “spacer” refers to a structure located between two moieties in a fusion protein that spatially separates the two moieties. Examples of spacers include but are not limited to amino acids, amino acid derivatives or analogues and amino acid sequences. Preferable spacers may include but are not limited to amino acid sequences comprising polymers of glycine (G), alanine (A) and serine (S), such as GA, GGG, GAG and (G)ₒS((G)ₛS)ₜ, wherein o is an integer of 1-5; s is an integer of 1-10, especially 4; t is 0 or an integer of 1-10. The spacer may be incorporated into a polypeptide (such as the antibody moiety or cytokine moiety as described herein) via chemical synthesis, chemical or enzymatic reaction or recombinant DNA technology, but not limited to.

As used herein, the terms “recognition sequence” and “recognition motif” are used interchangeably to refer to an amino acid sequence which is recognized by an enzyme (e.g., a Sortase) and on which the enzyme exerts its enzymatic action. For example, two polypeptides comprising the donor and acceptor substrate recognition sequences of a Sortase respectively can be conjugated to form a fusion polypeptide via the enzymatic action of the Sortase.

As used herein, “sequence identity” has an art-recognized meaning and the percent of sequence identity between two nucleic acids or polypeptides can be calculated by aligning the two sequences using published algorithms, such as the Basic Local Alignment Search Tool (BLAST) and the Fast Adaptive Shrinkage/Thresholding Algorithm (FASTA) (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994). While there are a number of methods to measure identity between two polynucleotides or polypeptides, the term “identity” is well known to skilled artisans (Carrillo, H. & Lipman, D., SIAM J Applied Math 48:1073 (1988)).

As used herein, a “variant” of a reference polypeptide (such as a wild-type IL21) comprises one or more amino acid additions, deletions, insertions and/or substitutions comparing to the reference polypeptide.

As used herein, “treating” a subject with a disease or condition means that administering or applying a composition, a procedure or a regimen to the subject in an attempt to cure the disease or condition, or to arrest, alleviate, ameliorate or eliminate the symptoms of the disease or condition. Hence the term “treatment” encompasses prophylaxis, therapy and/or cure. The term “Prophylaxis” refers to prevention of a potential disease and/or a prevention of worsening of symptoms or progression of a disease. As used herein, treatment also encompasses any pharmaceutical use of the fusion protein or pharmaceutical composition as provided herein.

As used herein, the definition of “subject” includes human and non-human subjects, such as experimental animals (e.g., mouse, rabbit, and rat and non-human primates), and preferably refers to human.

As used herein, a “therapeutic effect” means an effect resulting from treatment of a subject that alters, typically ameliorates or eliminates the symptoms of a disease or condition.

As used herein, a “therapeutically effective amount” refers to the quantity of an agent, compound, or composition containing one or more active agents that is at least sufficient to produce a therapeutic effect following administration to a subject. Hence, it is the quantity necessary for preventing, curing, ameliorating, arresting or partially arresting a symptom of a disease or disorder.

### Fusion Proteins

In an aspect, provided is a fusion protein having the structure of formula (I) or (I′):

[B-Sp₁-Lk-Sp₂]ₘ-A (I)

A-[Sp₁-Lk-Sp₂-B]ₘ (I′)
wherein
A is an anti-PD-L1 antibody;
B is an interleukin 21 (IL21);
Sp₁ and Sp2 are each independently a spacer or absent,
Lk is a linker formed by conjugation of an acceptor substrate recognition sequence and a donor substrate recognition sequence of a Sortase;
m is an integer of 1-4.

The fusion protein is an antibody-cytokine fusion protein (also referred to an immunocytokine) comprising an anti-PD-L1 antibody and an Interleukin 21 (IL21) conjugated through a linker, wherein the linker comprises an acceptor substrate recognition sequence conjugated with a donor substrate recognition sequence of a Sortase. In some embodiments, the linker is formed by Sortase-mediated site-specific conjugation of an acceptor substrate recognition sequence and a donor substrate recognition sequence recognized by the Sortase, wherein one of the recognition sequences is linked to the anti-PD-L1 antibody to form an antibody moiety, and the other one of the recognition sequences is linked to the IL21 to form a cytokine moiety.

In formula (I), the IL21 is linked to the donor substrate recognition sequence, and the anti-PD-L1 antibody is linked to the acceptor substrate recognition sequence. In formula (I′), the anti-PD-L1 antibody is linked to the donor substrate recognition sequence, and the IL21 is linked to the acceptor substrate recognition sequence. By the enzymatic action of the Sortase, the acceptor and donor substrate recognition sequences form the linker between the anti-PD-L1 antibody and the IL21. Optionally, a spacer is inserted in between the recognition sequence and the anti-PD-L1 antibody and/or the IL21 to provide a spatial separation.

Formulae (I) and (I′) define the linkages between the IL21 or the anti-PD-L1 antibody and the linker, respectively, but are not intended to limit the position at which the IL21 is conjugated to the anti-PD-L1 antibody. In any of formulae (I) and (I′), the IL21 can be conjugated to any position of the anti-PD-L1 antibody.

In some embodiments, the anti-PD-L1 antibody comprises a light chain and a heavy chain. In one embodiment, the IL21 is conjugated at the N-terminus of the light chain and/or heavy chain of the anti-PD-L1 antibody. In one embodiment, the IL21 is conjugated at the C-terminus of the light chain and/or heavy chain of the anti-PD-L1 antibody. In some embodiments, the IL21 is conjugated at the non-terminus position of the anti-PD-L1 antibody.

In some preferred embodiments, in formula (I), the IL21 is conjugated at the N-terminus of the light chain and/or heavy chain of the anti-PD-L1 antibody. In a particular embodiment, the IL21 is conjugated at the N-terminus of the heavy chain of the anti-PD-L1 antibody.

In some preferred embodiments, in formula (I′), the IL21 is conjugated at the C-terminus of the light chain and/or heavy chain of the anti-PD-L1 antibody. In a particular embodiment, the IL21 is conjugated at the C-terminus of the heavy chain of the anti-PD-L1 antibody.

m represents the number of the IL21 conjugated to the anti-PD-L1 antibody. Through controlling the number of donor or acceptor substrate recognition sequence linked to the anti-PD-L1 antibody, one or more IL21 can be conjugated to the anti-PD-L1 antibody. In some preferred embodiments, m is an integer selected from 1, 2, 3 and 4. In a particular embodiment, m is 1. In another particular embodiment, m is 2.

### Anti-PD-L1 Antibody

“Programmed cell death ligand 1 (PD-L1)”, also referred to as “B7H1” or “CD274”, is a ligand of the immunosuppressive receptor Programmed cell death 1 (PD-1). The PD-L1 may be from any species, such human, monkey, chimpanzee, cattle, pig, goat, sheep, horse, rat, mouse, guinea pig, rabbit and the like. Preferably, the PD-L1 is human PD-L1. The complete amino acid sequence of human PD-L1 (hPD-L1) is available in public databases, for example Uniprot and GenBank. The anti-PD-L1 antibody may be any antibody directed against PD-L1 (for example, PD-L1 comprising an amino acid sequence as shown in Uniprot identifier Q9NZQ7-1, Q9NZQ7-2 or Q9NZQ7-3) or fragments thereof. Preferably, the anti-PD-L1 antibody blocks the binding between PD-L1 and PD-1 and inhibits PD-L1 signaling. Exemplary anti-PD-L1 antibodies include, but are not limited to Atezolizumab (Tecentriq), Avelumab (Bavencio), Durvalumab (Imfinzi) or 3F2 (see WO 2020169062 A1, the content of which is incorporated herein by reference in its entirety). The anti-PD-L1 antibody may comprise further modifications. Modifications may include one or more amino acid additions, deletions, insertions and/or substitutions comparing to the unmodified antibody. The anti-PD-L1 antibody may be an engineered antibody prepared from Atezolizumab, Avelumab, Durvalumab or 3F2.

In some embodiments, the anti-PD-L1 antibody is a polyclonal antibody. In some embodiments, the anti-PD-L1 antibody is a monoclonal antibody. Methods for producing polyclonal and monoclonal antibodies are known in the art. For example, a fragment of PD-L1 as an antigen may be used to immunize a mammal, for instance, mouse, rat, rabbit, guinea pig, hamster, goat or sheep, and then polyclonal anti-PD-L1 antibodies may be purified from the serum of the immunized mammal; the anti-PD-L1 antibody-secreting B cells may be isolated from the immunized mammal and fused with myeloma cells to produce monoclonal anti-PD-L1 antibodies. Alternatively, a monoclonal anti-PD-L1 antibody may be generated using phage display technology and recombinant DNA technology.

In some embodiments, the anti-PD-L1 antibody is Atezolizumab, Avelumab, Durvalumab, 3F2 or an engineered antibody therefrom. In a particular embodiment, the anti-PD-L1 antibody is Atezolizumab or an engineered antibody therefrom. In another particular embodiment, the anti-PD-L1 antibody is anti-PD-L1 antibody 3F2 or an engineered antibody therefrom.

In some embodiments, the antibody comprises a light chain variable region (VL) and a heavy chain variable region (VH). In one embodiment, the VL comprises a CDRL1 sequence as shown in SEQ ID NO:22, a CDRL2 sequence as shown in SEQ ID NO:23 and a CDRL3 sequence as shown in SEQ ID NO:24, and the VH comprises a CDRH1 sequence as shown in SEQ ID NO:25, a CDRH2 sequence as shown in SEQ ID NO:26 and a CDRH3 sequence as shown in SEQ ID NO:27. In another embodiment, the VL comprises a CDRL1 sequence as shown in SEQ ID NO:28, a CDRL2 sequence as shown in SEQ ID NO:29 and a CDRL3 sequence as shown in SEQ ID NO:30, the VH comprises a CDRH1 sequence as shown in SEQ ID NO:31, a CDRH2 sequence as shown in SEQ ID NO:32 and a CDRH3 sequence as shown in SEQ ID NO:33.

In another embodiment, the VL comprises the amino acid sequence of SEQ ID NO:34, and the VH comprises the amino acid sequence of SEQ ID NO:35. In yet another embodiment, the VL comprises the amino acid sequence of SEQ ID NO:36, and the VH comprises the amino acid sequence of SEQ ID NO:37.

In some embodiments, the antibody comprises a light chain and a heavy chain. In one embodiment, the light chain comprises the amino acid sequence of SEQ ID NO:1, and the heavy chain comprises the amino acid sequence of SEQ ID NO:2. In another embodiment, the light chain comprises the amino acid sequence of SEQ ID NO:3, and the heavy chain comprises the amino acid sequence of SEQ ID NO:4.

The anti-PD-L1 antibody may be obtained through methods known in the art, including but not limited to phage display technology, hybridoma technology, recombinant DNA technology and chemical synthesis. The anti-PD-L1 antibody may be isolated from various sources, for example, from a yeast display library or a phage display library (see, e.g., Winter et al., (1994) Annu. Rev. Immunol. 12:433-455 and McCafferty et al., (1990) Nature 348:552-553) or human peripheral blood. For example, the antibody may be expressed in a host cell (e.g., yeast cells, bacteria, insect cells or mammalian cells) commonly used for antibody production. Mammalian host cells suitable for antibody expression include, but are not limited to, myeloma cells, Chinese Hamster Ovary (CHO) cells, HEK293 cells and other mammalian cells suitable for expressing antibodies. In a particular embodiment, a mammalian host cell is transformed expression vectors encoding the light chain and heavy chain of the anti-PD-L1 antibody, and the expressed antibodies are purified using a Protein A affinity column. Signal peptides may be used to help expression, correct folding and extracellular secretion of the antibody, thereby improving the quality, yield and purification of the antibody. Exemplary sequences of a signal peptide that can be added to the light chain and heavy chain are shown in SEQ ID NO:16 and SEQ ID NO:17, respectively.

### Interleukin 21 (IL21)

Interleukins 21 (IL21) is a 4α-helix bundle cytokine of approximately 14 kDa. IL21 signals through binding to a composite receptor consisting of IL21 receptor (IL21R) and the common cytokine γ-chain (gamma c). Without being bound to any theory, IL21 signaling induces proliferation, differentiation or activity of a variety of target immune cells including macrophages, B cells, CD4⁺T cells, CD8⁺T cells, cytotoxic T cells and natural killer cells, thereby enhancing both humoral and cell-mediated immune responses.

The IL21 may be from any species, for example, monkey, chimpanzee, cattle, pig, goat, sheep, horse, rat, mouse, guinea pig, rabbit and the like. Preferably, the IL21 is a human IL21 (hIL21). Two hIL21 precursors have been identified: UniProt ID: Q9HBE4-1 (SEQ ID NO:7, isoform 1 precursor) and UniProt ID: Q9HBE4-2 (SEQ ID NO:8, isoform 2 precursor), with the first 24 aa being the signal peptide. Wild-type IL21 is often secreted as a mature protein lacking the signal peptide. Exemplary amino acid sequences of a mature hIL21 are shown in SEQ ID NO:9 (isoform 1) and SEQ ID NO:10 (isoform 2).

The IL21 may be a wild-type IL21 or a variant thereof. A wild-type IL21 may have the amino acid sequence of SEQ ID NO:9 or SEQ ID NO:10. An IL21 variant may comprises one or more (for example, 1, 2, 3, 4, 5, 6, 7, 8, 9 or 10) amino acid substitutions, additions or deletions relative to SEQ ID NO:9 or SEQ ID NO:10. In some embodiments, the IL21 variant comprises an amino acid sequence having at least about 30%, 40%, 50%, 60%, 70%, 80%, 90%, 95%, 96%, 97%, 98% or 99% sequence identity to SEQ ID NO:9 or SEQ ID NO:10. In some embodiments, the IL21 variant binds to IL21R with a lower affinity comparing to the wild-type IL21. Examples of such IL21 variants may include IL21 muteins described in Shen et. al., Front. Immunol., (2020) 11:832; and WO 2019028316 A1, the relevant contents of which are incorporated herein by reference in their entirety.

The IL21 can be obtained through methods known in the art. For example, the coding sequence of IL21 may be generated through recombinant DNA technology and introduced into a suitable host cell for the expression of IL21. Exemplary host cells may include yeast cells, bacteria, fungi, insect cells and mammalian cells, but not limited to. In one embodiment, the IL21 is expressed in E. coli BL21 (DE3).

### Acceptor and Donor Substrate Recognition Sequences

The acceptor and donor substrate recognition sequences correspond to the type of the Sortase to be used. The Sortase can be a natural Sortase, an unnatural Sortase or a combination thereof. Natural Sortase may include Sortase A (SrtA), Sortase B (Srt B), Sortase C (SrtC), Sortase D (SrtD), Sortase E (SrtE), Sortase F (SrtF), etc. (see, e.g., US20110321183A1 and EP3647419A1).

A typical acceptor substrate recognition sequence includes an N-terminal amino group as a nucleophile, such as an N-terminal amino group of glycine and alanine. In some embodiments, the acceptor substrate recognition sequence comprises 1-100 series-connected structure units which are selected from the group consisting of glycine (G) and alanine (A). In a preferred embodiment, the acceptor substrate recognition sequence comprises the sequence of (G)ₙ, wherein n is an integer of 2-20, especially 3.

In some embodiments, the donor substrate recognition sequence is X₁X₂X₃TX₄X₅, wherein X₁ is leucine (L) or asparagine (N), X₂ is proline (P) or alanine (A), X₃ is any natural or unnatural amino acid, X₄ is glycine (G), serine (S) or asparagine (N) or absent, X₅ is an amino acid sequence comprising 1-10 amino acids or absent. In a preferred embodiment, X₃ is glutamic acid (E). In one embodiment, X₅ is absent. In another embodiment, X₅ is an amino acid sequence comprising 1-10 amino acids, wherein each amino acid is independently any natural or unnatural amino acid.

In some embodiments, the Sortase is a SrtA, such as SrtA from Staphylococcus aureus, X₁X₂X₃TX₄X₅ can be LPX₃TGG (SEQ ID NO:39), wherein X₃ is any natural or unnatural amino acid. In some embodiments, the Sortase is a SrtB, and X₁X₂X₃TX₄X₅ can be NPX₃TGG (SEQ ID NO:46), wherein X₃ is any natural or unnatural amino acid. In some embodiments, the Sortase is a SrtC, and X₁X₂X₃TX₄X₅ can be LPX₃TGG, wherein X₃ is any natural or unnatural amino acid. In some other embodiments, the Sortase is a SrtD, and X₁X₂X₃TX₄X₅ can be LPX₃TA (SEQ ID NO:47), wherein X₃ is any natural or unnatural amino acid. In yet some other embodiments, the Sortase is a SrtE, and X₁X₂X₃TX₄X₅ can be LAX₃TGG (SEQ ID NO:48), wherein X₃ is any natural or unnatural amino acid. In some other embodiments, the Sortase is a SrtF, and X₁X₂X₃TX₄X₅ can be LPX₃TG (SEQ ID NO:49), wherein X₃ is selected from the group consisting of A, R, E, N, D, Q, I, L and K. In some other embodiments, the Sortase is SrtA from Streptococcus pyogenes, and X₁X₂X₃TX₄X₅ can be LPX₃TGX₅ (SEQ ID NO:50), wherein X₅ is as defined above. In some embodiments, the Sortase is SrtE from Streptomyces coelicolor, and X₁X₂X₃TX₄X₅ can be LAX₃TG (SEQ ID NO:42). In yet another embodiment, the Sotase is SrtA from Lactobacillus plantarum, and X₁X₂X₃TX₄X₅ can be LPQTSEQ (SEQ ID NO:43). In one embodiment, the Sortase is SrtB from Staphylococcus aureus, and X₁X₂X₃TX₄X₅ can be NPQTN (SEQ ID NO:40). In another embodiment, the Sortase is SrtB from Bacillus anthracis, and X₁X₂X₃TX₄X₅ can be NPKTG (SEQ ID NO:41).

In some particular embodiments, the Sortase is SrtA from Staphylococcus aureus. Accordingly, X₁X₂X₃TX₄X₅ can be the typical donor substrate recognition sequence LPX₃TGG for the enzyme, wherein X₃ can be any single amino acid that is natural or unnatural. In some embodiments, X₁X₂X₃TX₄X₅ is LPX₃TGX₅, wherein X₃ is any single amino acid that is natural or unnatural; X₅ is absent or an amino acid sequence comprising 1-10 amino acids. In one embodiment, X₅ is absent. In another embodiment, X₅ is an amino acid sequence comprising 1-10 amino acids, wherein each amino acid is independently any natural or unnatural amino acid. In a particular embodiment, X₅ is (G) i, wherein i is an integer of 1 to 10. In another particular embodiment, X₁X₂X₃TX₄X₅ is LPETG (SEQ ID NO:44). In yet another particular embodiment, X₁X₂X₃TX₄X₅ is LPETGG (SEQ ID NO:45).

In a typical Sortase-catalyzed conjugation reaction, the Sortase cleaves the donor substrate recognition sequence X₁X₂X₃TX₄X₅ at the threonine residue. Upon cleavage, the fragment downstream of the threonine residue (-X₄X₅) is released; while the upstream fragment (X₁X₂X₃T-) forms a threonine-thioester with the active site cysteine of the Sortase to create an acyl-enzyme intermediate. The intermediate is then deacylated by ligating the sortase-bound thioester to the N-terminal amino group of the acceptor substrate recognition sequence. When (G)ₙ is used as the acceptor substrate recognition sequence, the linker Lk formed therefrom has the sequence of X₁X₂X₃T(G)ₙ, wherein X₁ is leucine (L) or asparagine (N), X₂ is proline (P) or alanine (A), X₃ is any natural or unnatural amino acid, n is an integer of 2-20, especially 3.

In some embodiments, X₁X₂X₃T(G), is LPX₃T(G)ₙ (SEQ ID NO:51), NPX₃T (G) n (SEQ ID NO:52) or LAX₃T (G)ₙ (SEQ ID NO:53), wherein L is leucine; P is proline; N is asparagine; A is alanine; X₃ is any natural or unnatural amino acid; T is threonine; G is glycine; and n is an integer of 2-20, especially 3. In one embodiment, n is selected from 2, 3, 4, 5, 6, 7, 8, 9, 10, 11, 12, 13, 14, 15, 16, 17, 18, 19 and 20. In a preferred embodiment, n is 3.

In some embodiments, X₁X₂X₃T(G)ₙ is LPX₃T(G)ₙ, wherein X₃ is any natural or unnatural amino acid, and n is as defined above. In one embodiment, X₃ is glutamic acid (E). In a particular embodiment, X₁X₂X₃T(G)ₙis LPETGGG (SEQ ID NO:54). In another embodiment, X₃ is glutamine (Q). In a particular embodiment, X₁X₂X₃T(G)ₙ is LPQTGGG (SEQ ID NO:55).

In yet another embodiment, X₁X₂X₃T(G)ₙ is LAX₃T(G)ₙ, wherein X₃ is any natural or unnatural amino acid, and n is as defined above. In a preferred embodiment, X₃ is E.

In another embodiment, X₁X₂X₃T(G)ₙ is NPQT(G)ₙ (SEQ ID NO:56) or NPKT(G)ₙ (SEQ ID NO:57), and n is as defined above.

### Spacers

In some embodiments, at least one of Sp₁ and Sp₂ is a spacer. In some embodiments, Sp₁ and Sp₂ are each independently a spacer.

In some embodiments, the spacer comprises 1-100 series-connected structure units which are selected from the group consisting of glycine (G), alanine (A) and serine (S). In some embodiments, the spacer is selected from the group consisting of GA, GGG, GAG and (G)ₒS((G)ₛS)ₜ (SEQ ID NO:59), wherein o is an integer of 1-5; s is an integer of 1-10, especially 4; t is 0 or an integer of 1-10. In a particular embodiment, Sp₁ is a spacer having the sequence of GA, and Sp₂ is a spacer having the sequence of GSGGGGS (SEQ ID NO:58).

### Specific Embodiments for the Fusion Protein

Exemplary embodiments of the fusion protein according to the present disclosure are provided as follows.

In some embodiments, Lk is X₁X₂X₃T(G)ₙ, and the fusion protein has the structure of (II) or (II′):

[B-Sp₁-X₁X₂X₃T(G)ₙ-Sp₂]ₘ-A (II)

A-[Sp₁-X₁X₂X₃T(G)ₙ-Sp₂-B]ₘ (II′)
wherein, X₁ is leucine (L) or asparagine (N), X₂ is proline (P) or alanine (A), X₃ is any natural or unnatural amino acid, n is an integer of 2-20,
m is 1 or 2;
A, B, Sp₁ and Sp₂ are as defined above.

In some embodiments, X₁X₂X₃T(G)ₙ is LPX₃T(G)ₙ. In one embodiment, X₃ is E. In another embodiment, X₃ is Q.

In some embodiments, X₁X₂X₃T(G)ₙ is LAX₃T(G)ₙ. In one embodiment, X₃ is E or Q.

In some embodiments, the anti-PD-L1 antibody is Atezolizumab, Avelumab, Durvalumab, 3F2 or an engineered antibody therefrom. In a particular embodiment, the anti-PD-L1 antibody is Ab0001 (Atezolizumab), which comprises a light chain having the amino acid sequence of SEQ ID NO:1 and a heavy chain having the amino acid sequence of SEQ ID NO:2. In another embodiment, the anti-PD-L1 antibody is Ab0002 (3F2), which comprises a light chain having the amino acid sequence of SEQ ID NO:3 and a heavy chain having the amino acid sequence of SEQ ID NO:4.

In some embodiments, the IL21 is a wild-type IL21 or a variant thereof. In one embodiment, the IL21 comprises the amino acid sequence of SEQ ID NO:9 or SEQ ID NO:10.

In one embodiment, in formula (II) or (II′), n is 3.

In some embodiments, Sp₁ and Sp₂ are absent. In some embodiments, at least one of Sp₁ and Sp₂ is a spacer as described herein. In some embodiments, Sp₁ and Sp₂ are each independently a spacer. In one embodiment, Sp₁ is GA, and Sp₂ is GSGGGGS.

In one embodiment, Sp₁ is GA, the donor substrate recognition sequence is LPETGG, the acceptor substrate recognition sequence is GGG, Sp₂ is GSGGGGS, and the fusion protein has the structure of (III) or (III′):

[B-GALPETGGGGSGGGGS]ₘ-A (III)

A-[GALPETGGGGSGGGGS-B]ₘ (III′)

GALPETGGGGSGGGGS is as shown in SEQ ID NO:60.

In one embodiment, in any one of formulae (I), (I′), (II), (II′), (III) and (III′), the anti-PD-L1 antibody comprises a light chain having the amino acid sequence of SEQ ID NO:1 and a heavy chain having the amino acid sequence of SEQ ID NO:2; or a light chain having the amino acid sequence of SEQ ID NO:3 and a heavy chain having the amino acid sequence of SEQ ID NO:4; and the IL21 comprises the amino acid sequence of SEQ ID NO:9 or SEQ ID NO:10; m is 1 or 2.

In some embodiments, in any one of formulae (I), (II) and (III), the anti-PD-L1 antibody comprises a light chain and a heavy chain. In one embodiment, the IL21 is conjugated at the N-terminus of the light chain and/or heavy chain of the anti-PD-L1 antibody. In a particular embodiment, the IL21 is conjugated at the N-terminus of the heavy chain of the anti-PD-L1 antibody.

In some other embodiments, in any one of formulae (I′), (II′) and (III′), the anti-PD-L1 antibody comprises a light chain and a heavy chain. In one embodiment, the IL21 is conjugated at the C-terminus of the light chain and/or heavy chain of the anti-PD-L1 antibody. In a particular embodiment, the IL21 is conjugated at the C-terminus of the heavy chain of the anti-PD-L1 antibody.

In some embodiments, the anti-PD-L1 antibody comprises two identical light chains and two identical heavy chains, wherein each light chain comprises the amino acid sequence of SEQ ID NO:1, and each heavy chain comprises the amino acid sequence of SEQ ID NO:2; or each light chain comprises the amino acid sequence of SEQ ID NO:3, and each heavy chain comprises the amino acid sequence of SEQ ID NO:4. In one embodiment, the IL21 is conjugated to the N-terminus of at least one of the heavy chains of the anti-PD-L1 antibody. In one embodiment, the fusion protein according to the present disclosure comprises two identical light chain moieties and two identical heavy chain moieties, wherein each heavy chain moiety comprises an IL21 conjugated to the N-terminus of each of the heavy chains of the anti-PD-L1 antibody. In another embodiment, the fusion protein according to the present disclosure comprises two identical light chain moieties and two different heavy chain moieties, wherein only one heavy chain moiety comprises an IL21 conjugated to one of the heavy chains of the anti-PD-L1 antibody.

In a particular embodiment, the fusion protein comprises two identical heavy chain moieties and two identical light chain moieties, and wherein
(1) each of the heavy chain moieties comprises the amino acid sequence of SEQ ID NO:18 or SEQ ID NO:20; and each of the light chain moieties comprises the amino acid sequence of SEQ ID NO:1; or
(2) each of the heavy chain moiety comprises the amino acid sequence of SEQ ID NO:19 or SEQ ID NO:21; and each of the light chain moieties comprises the amino acid sequence of SEQ ID NO:3.

In another particular embodiment, the fusion protein comprises two different heavy chain moieties and two identical light chain moieties, and wherein
(1) one of the heavy chain moieties comprises the amino acid sequence of SEQ ID NO:18 or SEQ ID NO:20, the other one of the heavy chain moieties comprises the amino acid sequence of SEQ ID NO:14; and each of the light chain moieties comprises the amino acid sequence of SEQ ID NO:1; or
(2) one of the heavy chain moieties comprises the amino acid sequence of SEQ ID NO:19 or SEQ ID NO:21, the other one of the heavy chain moieties comprises the amino acid sequence of SEQ ID NO:15; and each of the light chain moieties comprises the amino acid sequence of SEQ ID NO:3.

### Methods for Preparing the Fusion Protein

In some embodiments, the fusion protein is produced from conjugating an antibody moiety comprising an anti-PD-L1 antibody and a cytokine moiety comprising an IL21 under the catalysis of a Sortase, wherein one of the antibody moiety and the cytokine moiety further comprises a donor substrate recognition sequence of the Sortase, and the other one further comprises an acceptor substrate recognition sequence of the Sortase, such that the donor substrate recognition sequence and the acceptor substrate recognition sequence are conjugated via the enzymatic action of the Sortase. Suitable Sortases, acceptor and donor substrate recognition sequences are described above.

Accordingly, in another aspect, provided is a method for producing the fusion protein according to the present disclosure.

In one embodiment, the method comprises the following steps:
(a-1) providing an antibody moiety having the structure of formula (I-1) and a cytokine moiety having the structure of formula (I-2)

[Lkₐ-Sp₂]ₘ-A (I-1)

B-Sp₁-Lk_{d} (I-2);
and
(b-1) conjugating the antibody moiety with the cytokine moiety in the presence of a Sortase to obtain the fusion protein having the structure of formula (I)

[B-Sp₁-Lk-Sp₂]ₘ-A (I);
wherein
Lkₐ is an acceptor substrate recognition sequence of the Sortase, Lk_{d} is a donor substrate recognition sequence of the Sortase;
A, B, Sp₁, Sp₂, Lk and m are as defined above.

In another embodiment, the method comprises the following steps:
(a-2) providing an antibody moiety having the structure of formula (I′-1) and a cytokine moiety having the structure of formula (I′-2)

A-[Sp₁-Lk_{d}]ₘ (I′-1)

Lkₐ-Sp₂-B (I′-2);
and
(b-2) conjugating the antibody moiety with the cytokine moiety in the presence of a Sortase to obtain the fusion protein having the structure of formula (I′)

A-[Sp₁-Lk-Sp₂-B]ₘ (I′);
wherein
Lkₐ is an acceptor substrate recognition sequence of the Sortase, Lk_{d} is a donor substrate recognition sequence of the Sortase;
A, B, Sp₁, Sp₂, Lk and m are as defined above.

In one particular embodiment, step (a-1) further comprises:
(a-1-1) adding Lkₐ-Sp₂ to the N-terminus of the heavy chain of the anti-PD-L1 antibody to obtain the antibody moiety having the structure of formula (I-1)

[Lkₐ-Sp₂]ₘ-A (I-1);
and
(a-1-2) adding Sp₁-Lk_{d} to the C-terminus of an IL21 to obtain the cytokine moiety having the structure of formula (I-2)

B-Sp₁-Lk_{d} (I-2).

In another particular embodiment, step (a-2) further comprises:
(a-2-1) adding Sp₁-Lk_{d} to the C-terminus of the heavy chain of the anti-PD-L1 antibody to obtain the antibody moiety having the structure of formula (I′-1)

A-[Sp₁-Lk_{d}]ₘ (I′-1);

and
(a-2-2) adding Lkₐ-Sp₂ to the N-terminus of an IL21 to obtain the cytokine moiety having the structure of formula (I′-2)

Lkₐ-Sp₂-B (I′-2).

In one embodiment, Lkₐ comprises 1-100 series-connected structure units which are selected from the group consisting of glycine (G) and alanine (A). In a particular embodiment, Lkₐ comprises the sequence of (G)ₙ, wherein n is an integer of 2-20, especially 3.

In one embodiment, Lk_{d} comprises the sequence of X₁X₂X₃TX₄X₅, wherein, X₁ is L or N, X₂ is P or A, X₃ is any natural or unnatural amino acid, X₄ is G, S or N or absent, X₅ is an amino acid sequence comprising 1-10 amino acids or absent. In a particular embodiment, X₁X₂X₃TX₄X₅ is LPX₃TG (SEQ ID NO:38), LPX₃TGG, NPQTN, NPKTG, LAX₃TG or LPQTSEQ, wherein X₃ is any natural or unnatural amino acid. In a particular embodiment, X₁X₂X₃TX₄X₅ is LPETG or LPETGG.

In one embodiment, Sp₁ and Sp₂ are absent. In one embodiment, at least one of Sp₁ and Sp₂ is a spacer as described herein. In another embodiment, Sp₁ and Sp₂ are each independently a spacer. In some embodiment, the spacer comprises 1-100 series-connected structure units which are selected from the group consisting of G, A and S. In a preferred embodiment, the spacer is selected from the group consisting of GA, GGG, GAG and (G)ₒS((G)ₛS)ₜ, wherein o is an integer of 1-5; s is an integer of 1-10, especially 4; t is 0 or an integer of 1-10. In a particular embodiment, Sp₁ is a spacer having the sequence of GA, and Sp₂ is a spacer having the sequence of GSGGGGS.

In one embodiment, the anti-PD-L1 antibody is Atezolizumab, Avelumab, Durvalumab, 3F2 or an engineered antibody therefrom. In one embodiment, the anti-PD-L1 antibody comprises a VL and a VH, wherein the VL and VH comprise the CDR sequences as described herein. In another embodiment, the anti-PD-L1 antibody comprises a VL and a VH having the sequences as described herein. In a particular embodiment, the anti-PD-L1 antibody comprises a light chain having the amino acid sequence of SEQ ID NO:1 and a heavy chain having the amino acid sequence of SEQ ID NO:2, or a light chain having the amino acid sequence of SEQ ID NO:3 and a heavy chain having the amino acid sequence of SEQ ID NO:4.

In one embodiment, the IL21 is a wild-type IL21 or a variant thereof. In a particular embodiment, the IL21 comprises the amino acid sequence of SEQ ID NO:9 or SEQ ID NO:10.

### The Antibody Moiety

The antibody moiety may be generated through modifying the anti-PD-L1 antibody as described above with the acceptor substrate recognition sequence or the donor substrate recognition sequence described herein. The modification may occur at any position of the anti-PD-L1 antibody. In some embodiments, the modification occurs at the N-terminus and/or C-terminus of the anti-PD-L1 antibody, for example, at the N-terminus of the antibody light chain and/or at the N-terminus of the antibody heavy chain. In some embodiments, the modification occurs at the non-terminus position of the anti-PD-L1 antibody, for example, at the side chain of the antibody light chain and/or heavy chain.

In some embodiments, m is an integer selected from 1, 2, 3 and 4. In a particular embodiment, m is 1. In another particular embodiment, m is 2.

In one embodiment, (G)ₙ is introduced to the N-terminus of the light chain of the anti-PD-L1 antibody. In another embodiment, (G)ₙ is introduced to the N-terminus of the heavy chain of the anti-PD-L1 antibody. In yet another embodiment, (G)ₙ is introduced to the N-terminus of the light chain and heavy chain of the anti-PD-L1 antibody. (G)ₙ and the anti-PD-L1 antibody are as defined above.

In one embodiment, X₁X₂X₃TX₄X₅ is introduced to the C-terminus of the light chain of the anti-PD-L1 antibody. In another embodiment, X₁X₂X₃TX₄X₅ is introduced to the C-terminus of the heavy chain of the anti-PD-L1 antibody. In yet another embodiment, X₁X₂X₃TX₄X₅ is introduced to the C-terminus of the light chain and heavy chain of the anti-PD-L1 antibody. X₁X₂X₃TX₄X₅ and the anti-PD-L1 antibody are as defined above.

In some embodiments, the antibody moiety comprises a light chain, wherein the light chain may be: 1) the light chain of the anti-PD-L1 antibody (denoted as LC); 2) an N-terminally modified light chain, which comprises the light chain of the anti-PD-L1 antibody with (G)ₙ introduced to the N-terminus (denoted as LCNT); or 3) an N-terminally modified light chain, which comprises the light chain of the anti-PD-L1 antibody with (G)ₙ-Sp₂ introduced to the N-terminus (denoted as LCNT_{L}). In some other embodiments, the antibody moiety comprises a heavy chain, wherein the heavy chain may be: 1) the heavy chain of the anti-PD-L1 antibody (denoted as HC); 2) an N-terminally modified heavy chain, which comprises the heavy chain of the anti-PD-L1 antibody with (G)ₙ introduced to the N-terminus (denoted as HCNT); or 3) an N-terminally modified heavy chain, which comprises the heavy chain of the anti-PD-L1 antibody with (G)ₙ-Sp₂ introduced to the N-terminus (denoted as HCNT_{L}). By combining the light chain and heavy chain of the antibody moiety, 8 types of antibody moiety can be obtained, and m in formulae (I) and (I′) can be 1, 2, 3 and 4.

In a particular embodiment, the antibody moiety is Ab0030, which is derived from the antibody Ab0001. Ab0030 comprises a light chain of SEQ ID NO:1 and a heavy chain of SEQ ID NO:14, wherein the heavy chain comprises, from the N-terminus to C-terminus: the acceptor substrate recognition sequence GGG, the spacer GSGGGGS and the heavy chain of Ab0001 (SEQ ID NO:2).

In another particular embodiment, the antibody moiety is Ab0033, which is derived from the antibody Ab0002. Ab0033 comprises a light chain of SEQ ID NO:3 and a heavy chain of SEQ ID NO:15, wherein the heavy chain comprises, from the N-terminus to C-terminus: the acceptor substrate recognition sequence GGG, the spacer GSGGGGS and the heavy chain of Ab0002 (SEQ ID NO:4).

### The Cytokine Moiety

The cytokine moiety may be generated through modifying the IL21 as described above with the donor substrate recognition sequence or the acceptor substrate recognition sequence, respectively.

In some embodiments, the antibody moiety comprises an anti-PD-L1 antibody and an acceptor substrate recognition sequence, for example (G)ₙ, and the cytokine moiety comprises an IL21 and a donor substrate recognition sequence, for example X₁X₂X₃TX₄X₅. In some embodiments, the donor substrate recognition sequence is located at the C-terminus of the cytokine moiety. In some embodiments, the donor substrate recognition sequence is located at the N-terminus of the cytokine moiety. In some embodiments, the donor substrate recognition sequence is located at a non-terminus position of the cytokine moiety.

In some embodiments, the antibody moiety comprises an anti-PD-L1 antibody and a donor substrate recognition sequence, for example X₁X₂X₃TX₄X₅, and the cytokine moiety comprises an IL21 and an acceptor substrate recognition sequence, for example (G)ₙ. In some embodiments, the acceptor substrate recognition sequence is located at the N-terminus of the cytokine moiety. In some embodiments, the acceptor substrate recognition sequence is located at the C-terminus of the cytokine moiety. In some embodiments, the acceptor substrate recognition sequence is located at a non-terminus position of the cytokine moiety.

The antibody moiety and the cytokine moiety can be obtained through various methods known in the art, including but not limited to, chemical synthesis, recombinant DNA technology and a combination thereof. In a preferred embodiment, polynucleotides encoding the light chain and heavy chain of the antibody moiety are generated through recombinant DNA technology and introduced into a suitable host cell; and then the antibody moiety can be expressed in the host cell and purified from the host cell or the culture medium. The host cell can be stably or transiently transformed. Isolation and purification of the antibody moiety and the cytokine moiety may be performed using well-known techniques in the art. For example, the antibody moiety can be purified with a Protein A affinity column.

### Exemplary Fusion Proteins

In one embodiment, the cytokine moiety is a obtained by modifying the IL21 (SEQ ID NO:9 or SEQ ID NO:10) at the C-terminus with a spacer GA and a donor substrate recognition sequence LPETGG (SEQ ID NO:6), and the cytokine moiety comprises the amino acid sequence of SEQ ID NO:11 or SEQ ID NO:12.

In one embodiment, the antibody moiety is Ab0030, which comprises a light chain of SEQ ID NO:1 and a heavy chain of SEQ ID NO:14. Ab0030 is derived from the anti-PD-L1 antibody Ab0001 (Atezolizumab) by modifying the heavy chain of Ab0001 (SEQ ID NO:2) at the N-terminus with an acceptor substrate recognition sequence GGG and a spacer GSGGGGS (SEQ ID NO:5). In another particular embodiment, the antibody moiety is Ab0033, which comprises a light chain of SEQ ID NO:3 and a heavy chain of SEQ ID NO:15. Ab0033 is derived from the antibody Ab0002 (3F2) by modifying the heavy chain of Ab0002 (SEQ ID NO:4) at the N-terminus with an acceptor substrate recognition sequence GGG and a spacer GSGGGGS (SEQ ID NO:5).

According to some embodiments of the present disclosure, the sequences of the cytokine IL21 and the anti-PD-L1 antibody, and the sequences of the cytokine moiety and antibody moiety obtained therefrom are listed in the table below.

| | | | | Sequence(s) of |
|---|---|---|---|---|
| | Sequence(s) of | | | the Antibody |
| Antibody/ | the Antibody/ | Modification | Antibody Moiety/ | Moiety/ |
| Cytokine | Cytokine | at the terminal | Cytokine Moiety | Cytokine Moiety |
| Ab0001 | SEQ ID NO: 1 | —* | Ab0030 | SEQ ID NO: 1 |
| | SEQ ID NO: 2 | SEQ ID NO: 5 | | SEQ ID NO: 14 |
| Ab0002 | SEQ ID NO: 3 | —* | Ab0033 | SEQ ID NO: 3 |
| | SEQ ID NO: 4 | SEQ ID NO: 5 | | SEQ ID NO: 15 |
| hIL21 | SEQ ID NO: 9 | SEQ ID NO: 6 | Cytokine moiety derived | SEQ ID NO: 11 |
| isoform 1 | | | from hIL21 isoform 1 | |
| hIL21 | SEQ ID NO: | SEQ ID NO: 6 | Cytokine moiety derived | SEQ ID NO: 12 |
| isoform 2 | 10 | | from hIL21 isoform 2 | |

*“—” indicates no terminal modification.

Exemplary fusion proteins according to the present disclosure are listed in Table 2.

### Pharmaceutical Compositions

Also provided is a pharmaceutical composition comprising the fusion protein according to the present disclosure and a pharmaceutical acceptable carrier.

Examples of pharmaceutical acceptable carriers include, but are not limited to: diluents (e.g., lactose, microcrystalline cellulose and dextrose), binders and adhesives (e.g., acacia, gelatin, starch paste and carboxymethyl cellulose), lubricants (e.g., polyethylene glycol, calcium stearate and steric acid), disintegrants (e.g., starches, cellulose and cross linker polymers), preservatives, vehicles, glidants (e.g., corn starch), sweeteners (e.g., mannitol and saccharin), coating materials (e.g., povidone, ethyl cellulose and synthetic polymers), and plasticizers (e.g., castor oil, diacetylated monoglycerides, and polyethylene glycol), structure forming excipients (e.g., cetostearyl alcohol and mineral oils), preservatives (e.g., benzyl alcohol, propylparaben and sodium benzoate), antioxidants (e.g., butyl hydroxyl toluene and butyl hydroxyl anisole), solubilizers (e.g., lanolin and cholesterol), gelling agents (e.g., carboxyl methyl cellulose, hydroxyl propyl cellulose and xanthan gum), and emollients (e.g., glycerin, mineral oil, petrolatum and isopropyl palmitate), solvents (e.g., water, alcohol, acetic acid and syrups), buffers (e.g., phosphate buffers and acetate buffers), antimicrobial preservatives (e.g., benzyl alcohol, butylparaben, phenol and thiomersal), antioxidants (e.g., ascorbic acid, sodium bisulfate, thiourea and butyl hydroxy toluene), chelating agents (e.g., disodium EDTA, dihydroxy ethyl glycine and citric acid), and emulsifying agents (e.g., sodium lauryl sulfate, cetrimide and macrogol esters).

The pharmaceutical composition provided herein can be in various dosage forms, e.g., in solid, semi-solid, liquid, powder, aqueous, or lyophilized forms.

The pharmaceutical composition provided herein can be administered into a subject by any method known in the art, for example, by systemic or local administration. Routes of administration include, but are not limited to, parenteral (e.g., intradermal, intramuscular, intraperitoneal, intravenous, subcutaneous, or intracavity), topical, epidural, or mucosal (e.g. intranasal or oral). The exact dosage to be administered will depend on various factors, such as the therapeutic objectives, the route of administration, and the condition of the subject, for example, the patient's health, body weight, sex, diet, etc. Accordingly, it will be necessary for the therapist to titer the dosage of the pharmaceutical composition and modify the route of administration as required to obtain the optimal therapeutic effect. Generally, the dosage ranges for the administration of the pharmaceutical composition provided herein are those large enough to produce desired effects, in which the PD-L1-positive diseased cells are eliminated.

In a preferred embodiment, the pharmaceutical composition of the present disclosure can be administered into a human subject in a therapeutically effective amount. A therapeutic dose of the antibodies could be, for example, between preferably 0.1-25 mg/kg body weight per single therapeutic dose and most preferably between 0.1-10 mg/kg body weight for single therapeutic dose. In a particular embodiment, any fusion protein of the present disclosure could be formulated in accordance with conventional practice for administration by any suitable route and could generally be in a liquid form (e.g., a solution of the fusion protein in a sterile physiologically acceptable buffer) for administration by, for example, an intravenous, intraperitoneal, subcutaneous, or intramuscular route.

### Treatments

Further provided is a method for treating a disease in a subject in need thereof, comprising administering to the subject a therapeutically effective amount of the fusion protein or the pharmaceutical composition according to the present disclosure.

Also related is the fusion protein or the pharmaceutical composition of the present disclosure for use in treating a disease in a subject in need thereof.

The present disclosure also relates to the use of the fusion protein or the pharmaceutical composition according to the present disclosure for the manufacture of a medicament for treating a disease in a subject in need thereof.

In some embodiments, said subject is a mammal, for example, mouse, rat, rabbit, goat, sheep, pig, chimpanzee, ape, cattle and human. In some preferred embodiments, said subject is a human.

In some embodiments, said disease is a cancer. Preferably, the cancer is a PD-L1-positive cancer. In some embodiments, the cancer is selected from melanoma, ovarian cancer, breast cancer, Merkel cell carcinoma, lung cancer (including small cell lung cancer and non-small cell lung cancer (such as squamous and non-non-squamous non-small cell lung cancer)), renal cell cancer, bladder cancer, colon cancer, colon adenocarcinoma, colorectal cancer, head and neck squamous cell carcinoma, mesothelioma, virally induced cancers, cervical cancer, nasopharyngeal cancer, soft tissue sarcomas, hematological malignancies, gastric cancer, gastric or gastroesophageal junction (GEJ) adenocarcinoma, esophagus cancer, squamous cell carcinoma of the esophagus, endometrial carcinoma, primary mediastinal B-cell lymphoma (PMBCL), urothelial carcinoma, microsatellite instability-high (MSI-H) or a mismatch repair deficient (dMMR) solid tumor, hepatocellular carcinoma, lymphoma, Hodgkin's disease (Hodgkin lymphoma), non-Hodgkin's disease (non-Hodgkin's lymphoma), and diffuse large B-cell lymphoma. In some embodiments, the cancer is a malignant cancer selected from colorectal cancer, melanoma, renal cell carcinoma (RCC), cutaneous squamous cell carcinoma (cSCC), triple-negative breast cancer (TNBC), non-small cell lung cancer and bladder cancer. In some preferred embodiments, the cancer is selected from gastric cancer, breast cancer, colon cancer, colon adenocarcinoma, urothelial cancer, lung cancer, liver cancer, endometrial cancer, head and neck cancer and ovarian cancer.

Although the present disclosure has been described with reference to particular embodiments, it is to be understood that these embodiments are merely illustrative of the application and principles of the present disclosure. Modifications can be made without departing from the spirit and scope of the present disclosure.

### Beneficial Effects

The fusion protein according to the present disclosure achieves at least one of the following technical effects:
(1) specifically inhibiting PD-L1 signaling and activating IL21 signaling;
(2) effectively activating CD4⁺T cells; and
(3) having a higher anti-tumor activity and a lower toxicity.

Furthermore, conventional recombinant DNA technology is often used to generate antibody-cytokine fusion proteins (immunocytokines). However, the yield of a recombinant immunocytokine prepared using the conventional method can be extremely low when compared with that of a naked antibody (without conjugating to a cytokine), which hinders clinical application of many immunocytokines. Using the enzyme-mediated conjugation method according to the present disclosure, the fusion protein can be prepared in a very high yield and purity.

### EXAMPLES

Unless otherwise stated, commonly used molecular biology, biochemistry and cell biology techniques are utilized to prepare the modified or unmodified anti-PD-L1 antibody and IL21. Unless otherwise stated, the instruments and reagents used in the examples are commercially available. The reagents can be used directly without further purification.
Flow Cytometry: CytoFLEX S
HIC-HPLC: Butyl-HIC; mobile phase A: 25 mM PB, 2 M (NH₄)₂SO₄, pH 7.0; mobile phase B: 25 mM PB, pH 7.0; flow rate: 0.8 mL/min; acquisition time: 25 min; injection amount: 20 μg; column temperature: 25° C.; detection wavelength: 280 nm; sample injector temperature: 8° C.
SEC-HPLC: column: TSK-gel G3000 SWXL, TOSOH 7.8 mm ID×300 mm, 5 μm; mobile phase: 0.2 M KH₂PO₄, 0.25 M KCl, pH 6.2; flow rate: 0.5 mL/min; acquisition time: 30 min; injection volume: 50 μL; column temperature: 25° C.; detection wavelength: 280 nm; sample tray temperature: 8° C.
CHO was obtained from Thermo Fisher Scientific; pcDNA 3.3 was obtained from Life Technology; MabSelect Sure ProA was obtained from GE.
Optimized recombinant enzyme Sortase A derived from Staphylococcus aureus is prepared in E. coli.

### Example 1: Construction and Expression of the Antibody Moiety

### 1.1 Construction of Expression Plasmids for Ab0030 and Ab0033

Two antibody moieties Ab0030 and Ab0033 were prepared based on anti-PD-L1 antibodies Ab0001 (Atezolizumab) and Ab0002 (3F2), respectively. The amino acid sequences (SEQ ID NO:) of Ab0001 and Ab0002 are shown in Table 1A.

TABLE 1A

| Antibody | CDRL1 | CDRL2 | CDRL3 | CDRH1 | CDRH2 | CDRL3 | VL | VH | LC | HC |
|---|---|---|---|---|---|---|---|---|---|---|
| Ab0001 | 22 | 23 | 24 | 25 | 26 | 27 | 34 | 35 | 1 | 2 |
| Ab0002 | 28 | 29 | 30 | 31 | 32 | 33 | 36 | 37 | 3 | 4 |

Specifically, the heavy chain of Ab0001 (SEQ ID NO:2) and the heavy chain of Ab0002 (SEQ ID NO:4) were modified at the N-terminus by addition of a peptide sequence GGGGSGGGGS (SEQ ID NO:5; i.e., acceptor substrate recognition sequence with a spacer).

In order to facilitate the expression and extracellular secretion of the antibody moiety, coding sequences of a light chain signal peptide (SEQ ID NO:16) and a heavy chain signal peptide (SEQ ID NO:17) were added to the N-terminus of the coding sequences of the light chain and heavy chain, respectively.

Coding sequences of Ab0030 and Ab0033 were codon-optimized for efficient expression in CHO cells and cloned into the pcDNA 3.3 vector to obtain the plasmid pairs for each antibody moiety.

### 1.2 Expression of Ab0030 and Ab0033

The expression plasmids for Ab0030 and Ab0033 obtained in Example 1.1 were transiently transfected into CHO cells, with a mass ratio of 2:1. After fermentation, cell culture supernatants containing the antibodies were collected. The expression levels for Ab0030 and Ab0033 as determined by ELISA assay were 757.60 mg/L and 622.96 mg/L, respectively.

### 1.3 Purification of Ab0030 and Ab0033

The Protein A column (MabSelect Sure ProA, 20 cm×5.0 cm) was washed with the equilibrium buffer (50 mM Tris-HCl, 150 mM NaCl, pH 7.4). Cell culture supernatants from Example 1.2 were filtered with 0.22 μm filter and loaded onto the column. Once the sample loading was completed, the column was washed with 5-10 column volumes of equilibrium buffer. The antibodies were eluted and collected in 50 mM citrate buffer, pH 3.5. The pH of the antibody solution was adjusted by adding in 6% (v/v) of 1 M Tris pH 10.5 to 5.5-6.5. Finally, the buffer of the antibody solution was exchanged to equilibrium buffer through ultrafiltration. The concentrations of Ab0030 and Ab0033 were determined by using a UV spectrometer. Ab0030 and Ab0033 were aliquoted and stored at −80° C. The purified antibody moiety Ab0030 comprises a light chain of SEQ ID NO:1 and a heavy chain of SEQ ID NO:14. The purified antibody moiety Ab0033 comprises a light chain of SEQ ID NO:3 and a heavy chain of SEQ ID NO:15 (see Table 1B).

TABLE 1B

| | Sequences of the | Modification | Antibody | Sequences of the |
|---|---|---|---|---|
| Antibody | Antibody | at the terminal | Moiety | Antibody Moiety |
| Ab0001 | LC: SEQ ID NO: 1 | —* | Ab0030 | LC: SEQ ID NO: 1 |
| (Atezolizumab) | HC: SEQ ID NO: 2 | SEQ ID NO: 5 | | HC: SEQ ID NO: |
| | | | | 14 |
| Ab0002 | LC: SEQ ID NO: 3 | —* | Ab0033 | LC: SEQ ID NO: 3 |
| (3F2) | HC: SEQ ID NO: 4 | SEQ ID NO: 5 | | HC: SEQ ID NO: |
| | | | | 15 |

*“—” indicates no terminal modification

### 1.4 Characterization of Ab0030 and Ab0033

Ab0030 and Ab0033 were analyzed by SDS-PAGE electrophoresis. As expected, the molecular weights of the heavy chain and light chain measured on SDS-PAGE are about 50 kDa and about 25 kDa, respectively. SEC-HPLC analysis demonstrate that the percentages of Ab0030 and Ab0033 monomers are about 97.5% and 96.2%, respectively, fulfilling the requirements for the preparation of the fusion proteins.

The PD-L1-binding activity of Ab0030 and Ab0033 were determined by Enzyme-Linked Immunosorbent Assay (ELISA). Recombinant hPD-L1 was diluted in coating buffer (1×PBS) to 0.5 μg/mL and coated to an ELISA plate at 4° C. overnight. The plate was blocked with 5% slim milk powder in coating buffer at 37° C. for 1 h. Serial-diluted Ab0030 and Ab0033 were added to the plate, and the plate was then incubated at 37° C. for 1 h. Finally, 1:5000 diluted anti-human-IgG (H+L)-HRP was added. After an incubation at 37° C. for 1 h, TMB substrate was added for color development. The data was collected using a microplate reader.

The results are shown in FIG. 2: Ab0030 (FIG. 2A) and Ab0033 (FIG. 2B) can specifically bind to the antigen hPD-L1.

### Example 2: Construction and Expression of the Cytokine Moiety

Using overlapping PCR, a polynucleotide encoding a peptide sequence GALPETGG (SEQ ID NO:6, which comprises a donor substrate recognition sequence of the Sortase A (LPETGG) and a spacer GA) was added to the 3′ of a polynucleotide encoding hIL21 (amino acid sequence is shown in SEQ ID NO:9 or SEQ ID NO:10) to generate the polynucleotide encoding the cytokine moiety (amino acid sequence is shown in SEQ ID NO:11 or SEQ ID NO:12, respectively). In order to facilitate the expression and extracellular secretion of the cytokine moiety, the coding sequence of a signal peptide (SEQ ID NO:13) was added to the 5′ of the polynucleotide encoding SEQ ID NO:11 or SEQ ID NO:12. The purified cytokine moiety has the amino acid sequence of SEQ ID NO:11 or SEQ ID NO:12.

hIL21 was expressed in E. coli BL21 (DE3). After expression, recombinant hIL21 was purified by Ni-NTA column. Purified hIL21 was stored in 1×PBS buffer.

### Example 3: Preparation of the Fusion Protein

Fusion proteins were obtained by conjugating the antibody moiety Ab0030 and Ab0033 as prepared in Example 1 with the cytokine moiety as prepared in Example 2 through Sortase-mediated site-specific conjugation.

### Step 1) Pre-Treatment

Ab0030 and Ab0033 and the cytokine moiety were pre-treated using ultrafiltration membrane with suitable pore size and then concentrated, buffer-exchanged to conjugation buffer.

### Step 2) Enzyme-Mediated Conjugation

Ab0030 and Ab0033 (1-100 mg/mL) and the cytokine moiety (SEQ ID NO:11; 1-100 mg/mL) were separately prepared in the reaction buffer. The conjugation column which contains immobilized recombinant SrtA derived from Staphylococcus aureus was pre-treated with NaOH and high salt and pre-warmed to 10-40° C. in an air- or water-bath for more than 30 min. The reaction solutions containing the antibody moiety and the cytokine moiety were mixed at a fixed ratio and slowly pumped through the conjugation column to perform the conjugation reaction. Alternatively, the reaction solutions may be mixed with an immobilized recombinant SrtA or purified recombinant SrtA for 5 min to 24 h to perform the conjugation reaction.

The antibody-cytokine conjugation mixtures containing the fusion proteins were collected for further purification and DAR (DAR=the average number of hIL21 conjugated to the anti-PD-L1 antibody) determination.

### Step 3) Purification of the Fusion Protein

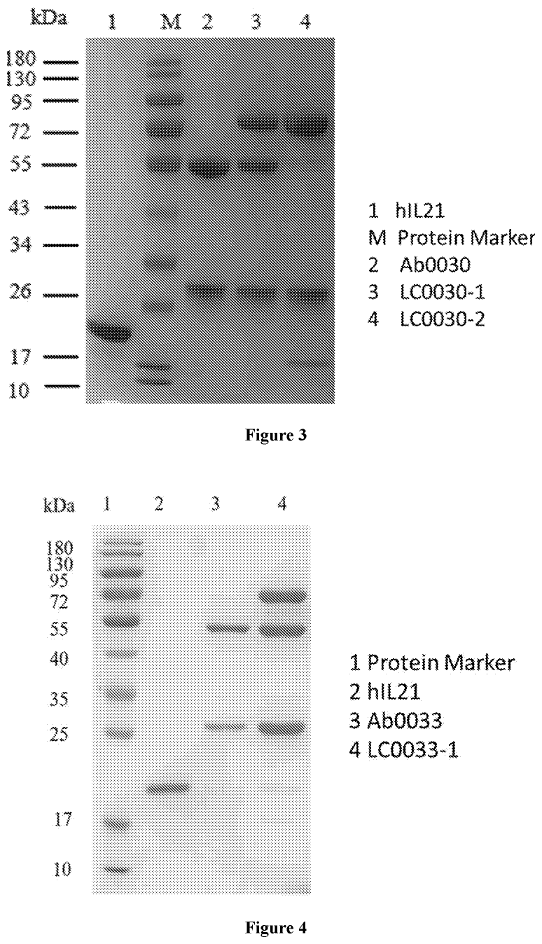

The fusion proteins were purified through cation exchange chromatography. Briefly, the chromatography column was packed with Capto S ImpAct medium and equilibrated with citric acid-sodium citrate buffer, pH 5.0-6.0. The conjugation mixtures from step 2) was loaded to the column. The column was flushed with citric acid-sodium citrate buffer, pH 5.0-6.0, until the desired offset (baseline) was reached. The unconjugated antibody moiety and cytokine moiety were eluted with citric acid-sodium citrate buffer, 100-500 mM NaCl, pH 5.0-6.0. The fusion proteins were eluted by increasing the NaCl concentration in citric acid-sodium citrate buffer (pH 5.0-6.0) from 0.5 M to 1 M. Unexpectedly, two fractions representing two different fusion protein populations were eluted in this step. Then the two fractions were subjected to SDS-PAGE analysis. It was confirmed that the first fraction represents a major population having a DAR value of 1 (referred to as DAR1 population, each fusion protein contains one hIL21) and the second fraction represents a minor population having a DAR value of 2 (referred to as DAR2 population, each fusion protein contain two hIL21). The fusion proteins containing only one hIL21 (isoform 1) are named as LC0030-1 (prepared from Ab0030) and LC0033-1 (prepared from Ab0033), respectively. The fusion proteins containing two hIL21 (isoform 1) are named as LC0030-2 prepared from Ab0030 and LC0033-2 prepared from Ab0033, respectively. FIG. 3 shows the SDS-PAGE analysis of the two fractions containing LC0030-1 and the LC0030-2, respectively. FIG. 4 shows the SDS-PAGE analysis of the fraction containing LC0033-1.

The column was regenerated using citric acid-sodium citrate buffer, 1 M NaCl, pH 5.0-6.0, and clean-in-place (CIP) was conducted using 1 M NaOH.

### Step 4) Ultrafiltration of the Fusion Protein

Purified fusion proteins were concentrated and buffer-exchanged to storage buffer using ultrafiltration membrane with suitable pore size, and then stored at 2-8° C. or −80° C.

A fusion protein containing hIL21 (isoform 2) (the cytokine moiety has the amino acid sequence of SEQ ID NO:12) was prepared using the same method as described above. The fusion proteins containing only one hIL21 (isoform 2) are named as LC0030′-1 (prepared from Ab0030) and LC0033′-1 (prepared from Ab0033), respectively. The fusion proteins containing two hIL21 (isoform 2) are named as LC0030′-2 (prepared from Ab0030) and LC0033′-2 (prepared from Ab0033), respectively.

Fusion proteins prepared are listed in Table 2.

TABLE 2

| | | | Number of |
|---|---|---|---|
| | | | hIL21 |
| Cytokine | | | conjugated to |
| Moiety | Antibody Moiety | Fusion Protein | the Antibody |
| Cytokine | Ab0030 | LC0030-1 | 1 |
| moiety derived | (LC: SEQ ID NO: 1; | (two identical light chain moieties: SEQ ID | |
| from hIL21 | HC: SEQ ID NO: 14) | NO: 1; | |
| isoform 1 | | heavy chain moiety 1: SEQ ID NO: 18; | |
| (SEQ ID NO: | | heavy chain moiety 2: SEQ ID NO: 14) | |
| 11) | | LC0030-2 | 2 |
| | | (two identical light chain moieties: SEQ ID | |
| | | NO: 1; | |
| | | two identical heavy chain moieties: SEQ ID | |
| | | NO: 18) | |
| | Ab0033 | LC0033-1 | 1 |
| | (LC: SEQ ID NO: 3; | (two identical light chain moieties: SEQ ID | |
| | HC: SEQ ID NO: 15) | NO: 3; | |
| | | heavy chain moiety 1: SEQ ID NO: 19; | |
| | | heavy chain moiety 2: SEQ ID NO: 15) | |
| | | LC0033-2 | 2 |
| | | (two identical light chain moieties: SEQ ID | |
| | | NO: 3; | |
| | | two identical heavy chain moieties: SEQ ID | |
| | | NO: 19) | |
| Cytokine | Ab0030 | LC0030′-1 | 1 |
| moiety derived | (LC: SEQ ID NO: 1; | (two identical light chain moieties: SEQ ID | |
| from hIL21 | HC: SEQ ID NO: 14) | NO: 1; | |
| isoform 2 | | heavy chain moiety 1: SEQ ID NO: 20; | |
| (SEQ ID NO: | | heavy chain moiety 2: SEQ ID NO: 14) | |
| 12) | | LC0030′-2 | 2 |
| | | (two identical light chain moieties: SEQ ID | |
| | | NO: 1; | |
| | | two identical heavy chain moieties: SEQ ID | |
| | | NO: 20) | |
| | Ab0033 | LC0033′-1 | 1 |
| | (LC: SEQ ID NO: 3; | (two identical light chain moieties: SEQ ID | |
| | HC: SEQ ID NO: 15) | NO: 3; | |
| | | heavy chain moiety 1: SEQ ID NO: 21; | |
| | | heavy chain moiety 2: SEQ ID NO: 15) | |
| | | LC0033′-2 | 2 |
| | | (two identical light chain moieties: SEQ ID | |
| | | NO: 3; | |
| | | two identical heavy chain moieties: SEQ ID | |
| | | NO: 21) | |

### Example 4 HIC-HPLC Analysis of the Fusion Protein

The fusion proteins as prepared in Example 3 were subjected to Butyl-HIC HPLC analysis to determine the DAR distribution.

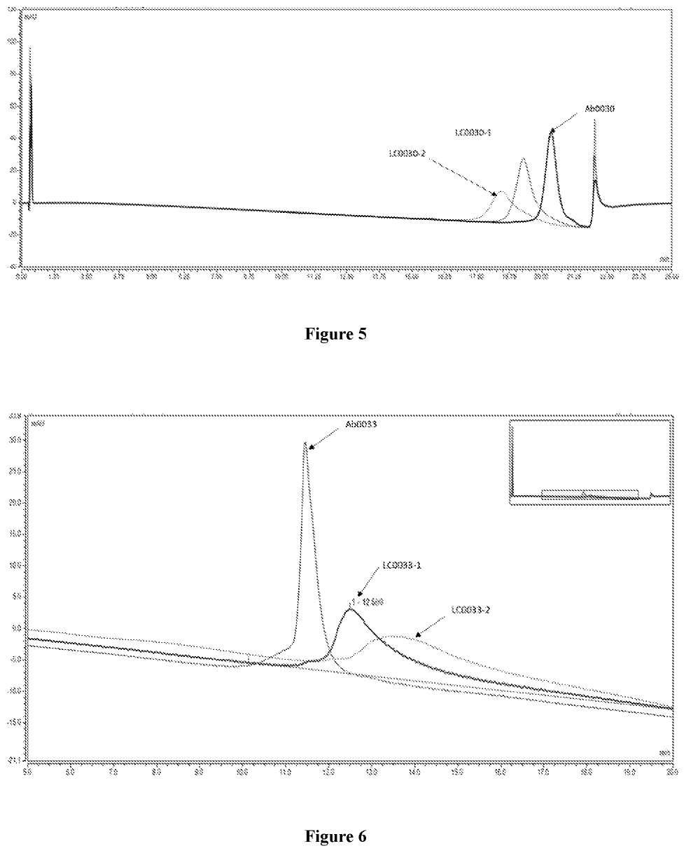

The results are shown in FIG. 5 and FIG. 6. The peak position of LC0030-1 shifted forward comparing to that of Ab0030, and the peak position of LC0030-2 shifted forward comparing to that of LC0030-1, indicating that the hydrophobicity of fusion proteins LC0030-1 and LC0030-2 are lower than that of Ab0030 (FIG. 5). Interestingly, the peak position of LC0033-1 shifted backward comparing to that of Ab0033, and the peak position of LC0033-2 shifted backward comparing to that of LC0033-1, indicating that the hydrophobicity of LC0033-1 and LC0033-2 are stronger than that of Ab0033 (FIG. 6). These differences may be caused by the amino acid and structure difference between Ab0030 and Ab0033.

### Example 5 Determining the Binding Activity of the Fusion Proteins to hPD-L1

The binding activities of the fusion proteins to human PD-L1 (hPD-L1) were determined using ELISA. Recombinant hPD-L1 protein (Acrobiosystems, PD1-H5253) were diluted with coating buffer (1×PBS) to 0.5 μg/mL and coated to an ELISA plate at 4° C. overnight. The plate was blocked with 5% slim milk powder in the coating buffer at 37° C. for 1 h. Serial-diluted Ab0030, Ab0033, LC0030-1 and LC0033-1 were added, and the plate was then incubated at 37° C. for 1 h. Finally, 1:5000 diluted anti-human-IgG (H+L)-HRP was added. After an incubation at 37° C. for 1 h, TMB substrate was added for color development. The data was collected using a microplate reader.

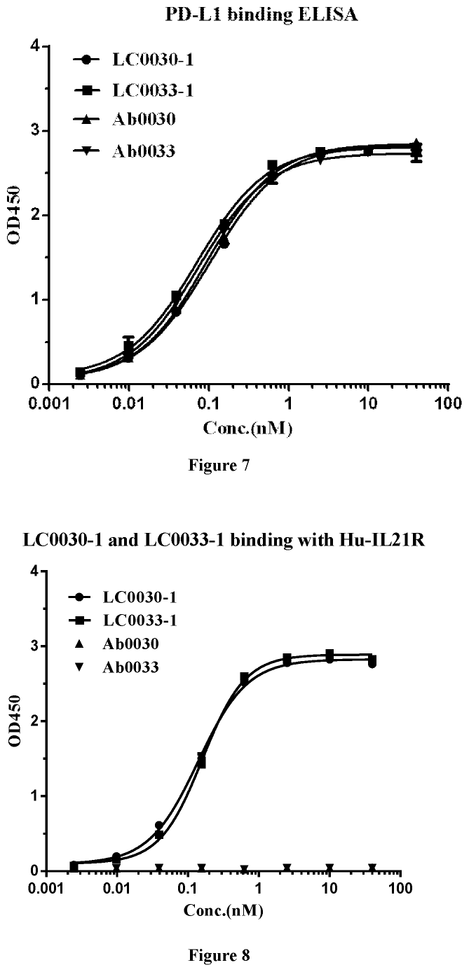

The results are shown in FIG. 7: both LC0030-1 and LC0033-1 can specifically bind to the antigen PD-L1 with a similar affinity to Ab0030 and Ab0033, indicating that the PD-L1-binding activities of the anti-PD-L1 antibodies in the fusion proteins are retained.

### Example 6 Determining the Binding Activity of the Fusion Proteins to the IL21 Receptor (IL21R)

The binding activities of the fusion proteins to human IL21 receptor (hIL21R or Hu-IL21R) were determined using ELISA. Recombinant hIL21R protein were diluted using coating buffer (1×PBS) to 0.5 μg/mL and coated to an ELISA plate at 4° C. overnight. The plate was blocked with 5% slim milk powder in coating buffer at 37° C. for 1 h. Serial-diluted Ab0030, Ab0033, LC0030-1 and LC0033-1 were added to the plate, and the plate was then incubated at 37° C. for 1 h. Finally, 1:5000 diluted anti-human-IgG (H+L)-HRP was added. After an incubation at 37° C. for 1 h, TMB substrate was added for color development. The data was collected using a microplate reader.

The results are shown in FIG. 8: LC0030-1 and LC0033-1 can specifically bind to hIL21R, while Ab0030 and Ab0033 do not bind to hIL21R. As the anti-human-IgG (H+L)-HRP binds to the human Fc portion of the antibody, the results suggest the successful conjugation of the anti-PD-L1 antibodies to hIL21 and the specific binding between the fusion proteins and hIL21R.

### Example 7 Determining the Binding Affinity of the Fusion Proteins to hPD-L1 and hIL21R

Surface plasmon resonance (SPR) was performed using Biacore (Biacore, INC, Piscataway N.J.) to measure the binding affinity of LC0033-1 and Ab0033 to hPD-L1 or hIL21R. The Biacore T200 biosensor is equipped with Sensor Chip Protein A. The proteins (diluted to 1 μg/mL) to be tested were injected at 10 μL/min into the Biacore T200 biosensor for 50 sec, in order to reach the required protein density (about 300 RU). Then His-tagged human IL21R (hIL21R-His, 200 nM) or human PD-L1 (hPD-L1-His, 100 nM) were injected at 30 μL/min into the system for 180 sec. The dissociation was monitored for 600 sec. After each cycle, glycine buffer pH 1.7 (30 μL/min, 30 sec) was used to regenerate the chip.

Data were processed with Biacore T200 evaluation software 3.0. The binding kinetics were analyzed by globally fitting the data with the 1:1 Langmuir binding model to obtain the association rate (kₒₙ) and dissociation rate (k_{off}). The affinity (K_{D}) was calculated using the formula: K_{D}=k_{off}/kₒₙ.

The results are shown in Table 3: LC0033-1 and Ab0033 can bind to hPD-L1-His with similar affinity. LC0033-1 can bind to IL21R with high affinity.

TABLE 3

| | | Association | Dissociation | |
|---|---|---|---|---|
| | | rate | rate | Affinity |
| Protein | Analyte | kₒₙ (1/Ms) | k_{off} (1/s) | K_{D} (M) |
| Ab0033 | hIL21R-His | N/A | N/A | N/A |
| LC0033-1 | hIL21R-His | 5.66E+05 | 2.77E−04 | 4.90E−10 |
| Ab0033 | hPD-L1-His | 1.98E+06 | 1.20E−03 | 6.08E−10 |
| LC0033-1 | hPD-L1-His | 6.62E+05 | 9.84E−04 | 1.48E−09 |

### Example 8 Determining the Ability of the Fusion Proteins to Activate CD4⁺T Cells

In vitro PBMC cell-stimulating activity of LC0033-1 was determined using a STAT3 phosphorylation (pSTAT3) assay. STAT3 phosphorylation is an indicator of CD4⁺T cell stimulation. Commercial PBMC cells (AllCells, Cat#FPB003F-C) were thawed and recovered at 37° C., and then suspended in pre-warmed culture media (RPMI 1640) and centrifuged at 300 g for 3 min. The suspension and centrifugation process was repeated three times. The cells were then added to 96-well plate at 10⁵ cells/well in 100 μL medium. Subsequently, serial diluted Ab0033, LC0033-1 or hIL21 were added into each well (an equal volume of PBS was added to the control well as a blank control) 100 μL and incubated at 37° C. for 30 min. An equal volume of fixative was added to the cells to a working concentration of 4% formaldehyde in PBS, and the cells were fixed at room temperature for 15 min. The cells were centrifuged at 300 g for 3 min to remove the supernatant and washed with 200 μL PBS. For intracellular staining, the cells were re-suspended in 100% ice-cold methanol and incubated for 30 min on ice. After a 3 min centrifugation at 300 g, the cells were washed with PBS+0.5% BSA for three times. 5 μL directly labeled antibody (Phospho-STAT3 (Tyr705) Monoclonal Antibody (LUVNKLA), APC, eBioscience™, #17-9033-42 and CD4 Monoclonal Antibody (RPA-T4), FITC, eBioscience™, #11-0049-42) was added to each well for staining. After a 30 min incubation at 4° C., the cells were analyzed using a Sartorius-IntelliCyt® iQue flow cytometer.

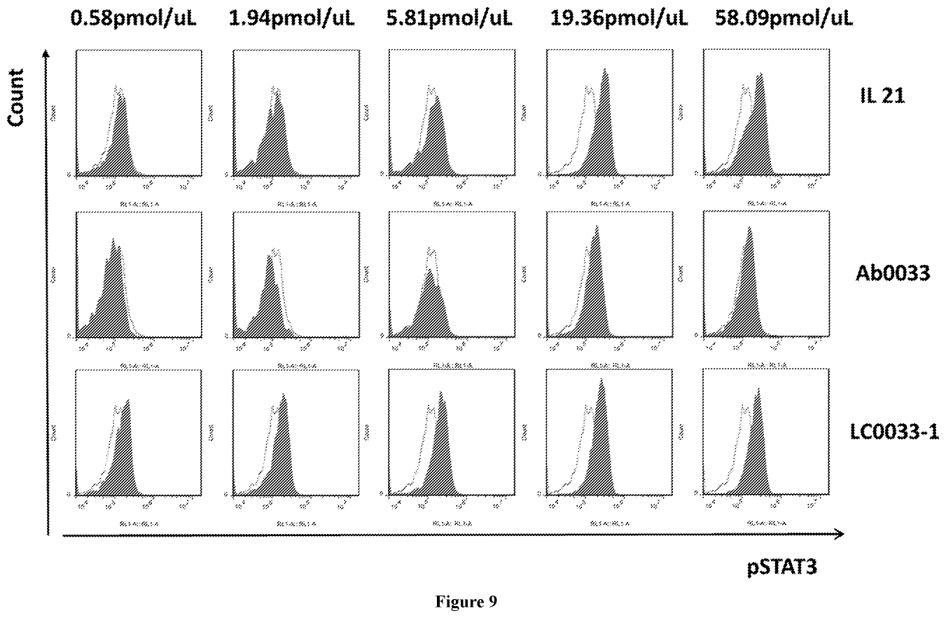

The results are shown in FIG. 9: comparing to the blank control (open histograms), the peaks of LC0033-1 and hIL21 (filled histograms) are obviously shifted to the right, indicating the two molecules are effective in inducing the phosphorylation of STAT3 in CD4⁺T cells and thus effective in CD4⁺T cell stimulation.

### Example 9 Evaluation of the In Vivo Efficacy of the Fusion Protein

To evaluate the in vivo efficacy of the fusion protein according to the present disclosure and to predict the therapeutic effects in human, humanized PD-1/PD-L1 mice (PD-1/PD-L1 gene humanized mice; see WO 2018041118 A1 and WO 2018001241 A1, the relevant contents of which are incorporated herein by reference in their entireties) were used to establish a mouse model of colon adenocarcinoma.

Two in vivo efficacy studies were performed using homozygotes of the above-mentioned humanized PD-1/PD-L1 mice (6-7 weeks) (Biocytogen Pharmaceuticals (Beijing) Co., Ltd., Cat#120522). Briefly, the mice were subcutaneously inoculated with murine colon adenocarcinoma cells MC38-hPD-L1 (5×10⁵/100 μL PBS) (Biocytogen Pharmaceuticals (Beijing) Co., Ltd.). When the tumors were about 100-150 mm³, the mice were randomly assigned into treatment or control groups based on their tumor volumes. The treatment group mice were randomized for administration of Ab0033, hIL21, LC0033-1, LC0033-2 or a combination of hIL21 and Ab0033 (equimolar). The control group mice were injected with an equal volume of saline. The mice were administrated (intravenously (i.v.), the first efficacy study; or intraperitoneally (i.p.), the second efficacy study) twice a week (BIW), 6 administrations in total. Tumor volumes and body weights of the mice were measured twice a week. When the tumor volume of an individual mouse reached 3000 mm³, the mouse was euthanized and the experiment was ended. The administration regimens for the two studies are summarized in Table 4 and 6, respectively.

### 9.1 The First Efficacy Study

The administration regimen for the first efficacy study is shown in Table 4.

TABLE 4

| | | | | | | Total |
|---|---|---|---|---|---|---|
| | | | | | | No. of |
| | No. of | Treat- | Dosage | | Fre- | adminis- |
| Group | Animals | ment | (mg/kg) | Route | quency | tration |
| G1 | 8 | Saline | — | i.v. | BIW | 6 |
| | | (control) | | | | |
| G2 | 8 | Ab0033 | 0.91 | i.v. | BIW | 6 |
| G3 | 8 | hIL21 | 0.091 | i.v. | BIW | 6 |
| G4 | 8 | Ab0033 | 2.73 | i.v. | BIW | 6 |
| G5 | 8 | hIL21 | 0.273 | i.v. | BIW | 6 |
| G6 | 8 | LC0033-1 | 1 | i.v. | BIW | 6 |
| G7 | 8 | Ab0033 + | 0.91 + 0.091 | i.v. | BIW | 6 |
| | | hIL21 | | | | |
| G8 | 8 | LC0033-1 | 3 | i.v. | BIW | 6 |
| G9 | 8 | Ab0033 + | 2.73 + 0.273 | i.v. | BIW | 6 |
| | | hIL21 | | | | |
| G10 | 8 | LC0033-2 | 1.1 | i.v. | BIW | 6 |

Tumor volumes at the day of grouping (Day 0) and 14 (Day 14) or 26 (Day 26) days after grouping were summarized in Table 5. The tumor growth inhibition value (TGI_{TV} %) was calculated using the formula: TGI_{TV}(%)=[1−(Tᵢ−T₀)/(Vᵢ−V0)]×100. Tᵢ is the average tumor volume in the treatment group on day i. T₀ is the average tumor volume in the treatment group on day zero. Vᵢ is the average tumor volume in the control group on day i. V₀ is the average tumor volume in the control group on day zero.

TABLE 5

| | Tumor volume (Mean ± SEM, mm³) | | | |
|---|---|---|---|---|
| | Day 0 | Day 14 | Day 26 | TGI_{TV} % |
| G1 | 104 ± 3 | 1326 ± 60 | 2764 ± 232 | |
| G2 | 104 ± 3 | 982 ± 96 | 2613 ± 399 | 5.7 |
| G3 | 104 ± 3 | 1013 ± 100 | 2410 ± 287 | 13.3 |
| G4 | 105 ± 3 | 1387 ± 192 | 2619 ± 601 | 5.5 |
| G5 | 104 ± 3 | 1078 ± 117 | 2572 ± 394 | 7.2 |
| G6 | 104 ± 4 | 780 ± 132 | 1155 ± 149 | 60.5 |
| G7 | 104 ± 3 | 852 ± 117 | 1820 ± 291 | 35.5 |
| G8 | 104 ± 3 | 584 ± 97 | 905 ± 172 | 69.9 |
| G9 | 105 ± 3 | 841 ± 47 | 2155 ± 199 | 22.9 |
| G10 | 105 ± 3 | 688 ± 92 | 1270 ± 314 | 56.2 |

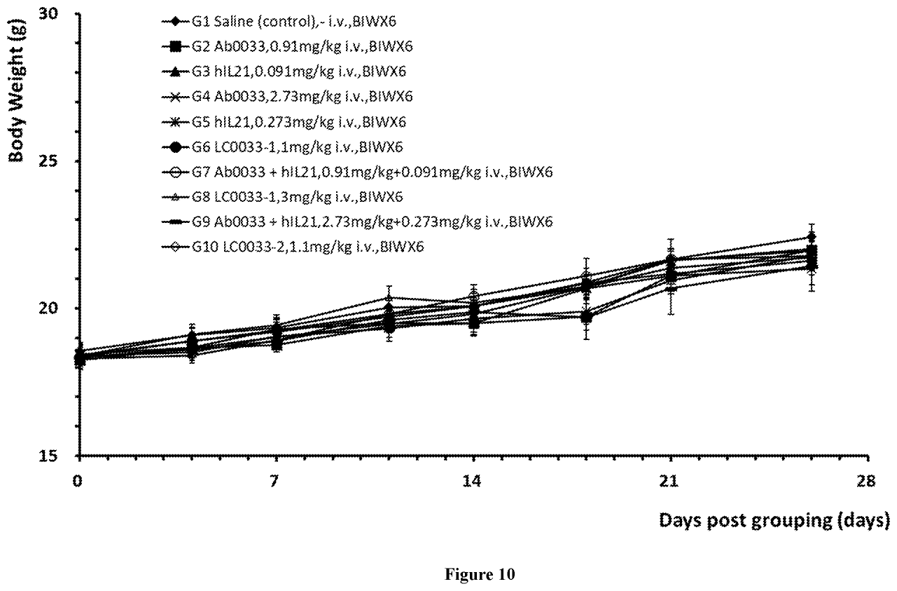

Overall, the mice in each group were in good health during the experiment. The treatment groups and the control group showed no significant difference in the body weight and change of body weight (FIG. 10). At the end of the experiment, the body weights of the mice in each group increased, and there was no significant difference in the body weight between the treatment groups and the control group, indicating that the drugs were well tolerated. The results indicate that LC0033-1, LC0033-2 and Ab0033 are safe and have no significant adverse effects to the animals.

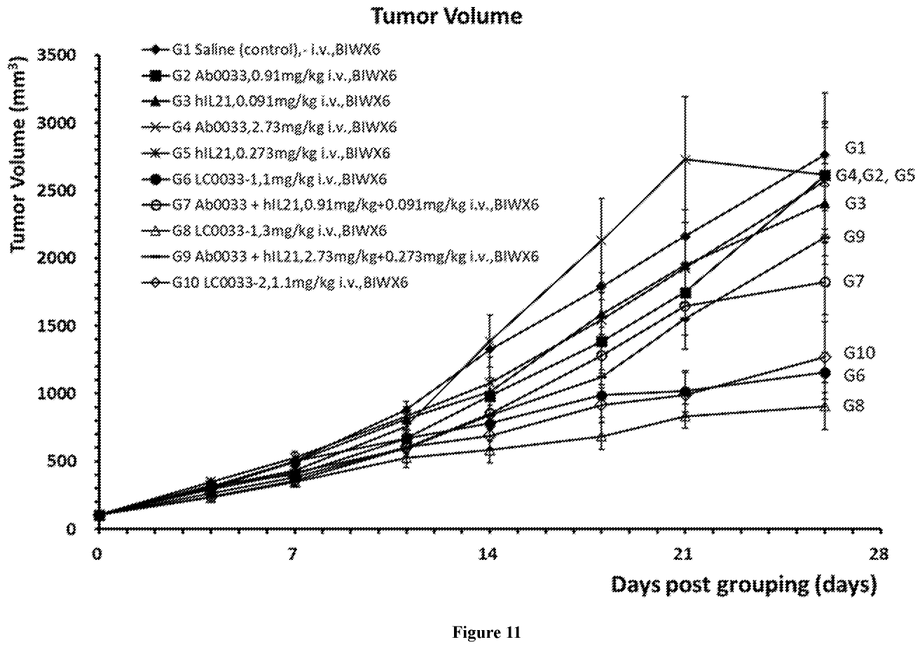

As seen from FIG. 11, tumors in the control mice continued to grow during the experiment, while tumor growth in the mice from the treatment groups were inhibited to different extents. In particular, tumor growth in the LC0033-1 and LC0033-2 groups (G6, G8 and G10) was significantly inhibited compared to the other treatment groups (G2, G3, G4, G5, G7 and G9), indicating that LC0033-1 and LC0033-2 exhibited superior in vivo tumor inhibitory activities compared to monotherapy with Ab0033 or hIL21 or their combination therapy.

As seen from Table 5 and FIG. 11, at the end point of the experiment, tumor volumes of all the mice treated with LC0033-1 (G6 and G8) or LC0033-2 (G10) were significantly smaller than those of the control mice (G1), indicating that LC0033-1 and LC0033-2 have tumor inhibitory effects at a low dosage. Furthermore, LC0033-1 and LC0033-2 exhibited similar efficacies (TGI_{TV} %: 60.5% versus 56.2%) at the same dosage. When the dosage of LC0033-1 increased from 1 mg/kg to 3 mg/kg, the TGI_{TV} % was increased from 60.5% to 69.9%, indicating a slight increase in the tumor inhibitory effect. Comparing to the significant anti-tumor activity of the fusion proteins (G8 and G10), the TGI_{TV} % from the groups treated with Ab0033 only, hIL21 only or the combination of Ab0033 and hIL21 was less than 25% (except G7), indicating that Ab0033, hIL21 and their combination did not exhibit significant anti-tumor activity.

### 9.2 The Second Efficacy Study

The administration regimen for the second efficacy study is shown in Table 6.

TABLE 6

| | | | | | | Total |
|---|---|---|---|---|---|---|
| | | | | | | No. of |
| | No. of | Treat- | Dosage | | Fre- | adminis- |
| Group | animals | ment | (mg/kg) | Route | quency | tration |
| G1 | 8 | Saline | — | i.p. | BIW | 6 |
| | | (control) | | | | |
| G2 | 8 | Ab0033 | 2.73 | i.p. | BIW | 6 |
| G3 | 8 | Ab0033 | 5.46 | i.p. | BIW | 6 |
| G4 | 8 | Ab0033 | 9.1 | i.p. | BIW | 6 |
| G5 | 7 | LC0033-1 | 3 | i.p. | BIW | 6 |
| G6 | 7 | LC0033-1 | 6 | i.p. | BIW | 6 |
| G7 | 7 | LC0033-1 | 10 | i.p. | BIW | 6 |

Tumor volumes at the day of grouping (Day 0) and 14 (Day 14) or 24 (Day 24) days after grouping were summarized in Table 7, and the tumor growth inhibition value (TGI_{TV} %) was calculated.

TABLE 7

| | Tumor volume (Mean ± SEM, mm³) | | | |
|---|---|---|---|---|
| | Day 0 | Day 14 | Day 24 | TGI_{TV} % |
| G1 | 123 ± 3 | 1162 ± 73 | 2461 ± 211 | N/A |
| G2 | 123 ± 3 | 685 ± 105 | 1525 ± 227 | 40.0 |
| G3 | 123 ± 3 | 890 ± 114 | 1926 ± 192 | 22.9 |
| G4 | 122 ± 4 | 410 ± 83 | 493 ± 126 | 84.1 |
| G5 | 123 ± 4 | 370 ± 29 | 583 ± 58 | 80.3 |
| G6 | 123 ± 4 | 458 ± 77 | 466 ± 72 | 85.3 |
| G7 | 123 ± 4 | 248 ± 38 | 385 ± 82 | 88.8 |

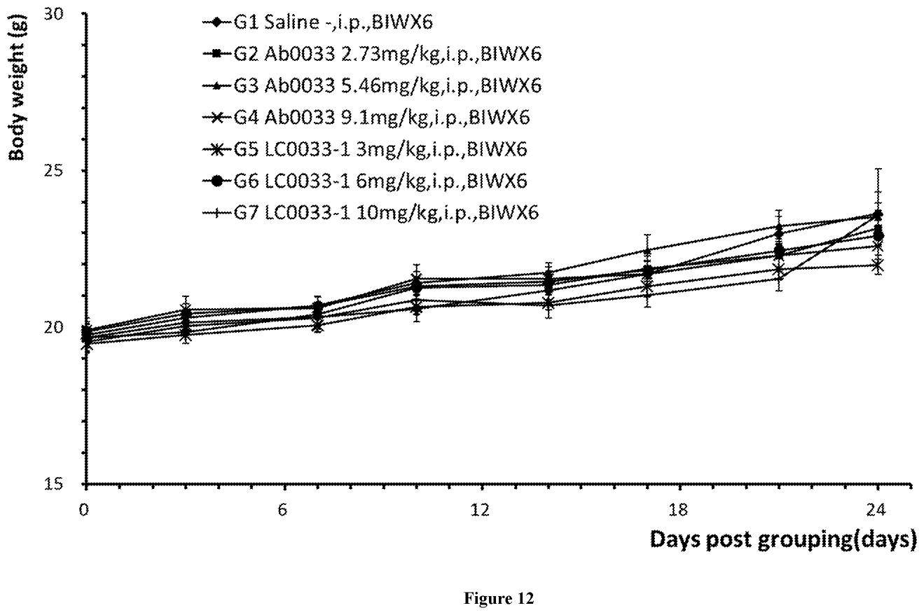

Overall, the mice in each group were in good health during the experiment. The treatment groups and the control group showed no significant difference in the body weight and change of body weight (FIG. 12). At the end of the experiment, the body weights of the mice in each group increased, and there was no significant difference in the body weight between the treatment groups and the control group, indicating the drugs were well tolerated. The results indicate that LC0033-1 and Ab0033 are safe and have no significant adverse effects to the animals.

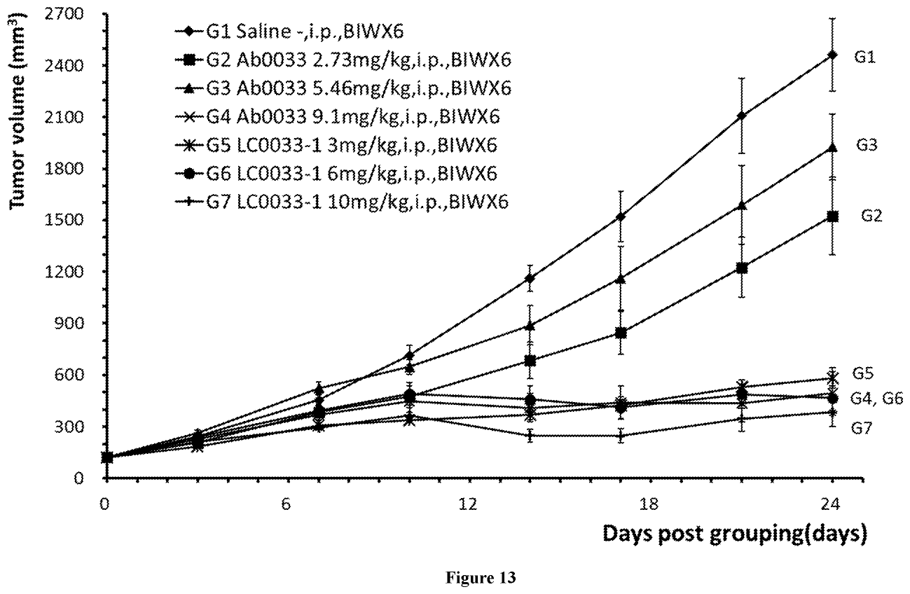

As seen from Table 7 and FIG. 13, tumors in the control mice continued to grow during the experiment, while tumor growth in the mice from the treatment groups were inhibited to different extents. At the end of the experiment (Day 24), the tumor volumes of the mice from G2-G7 are smaller than those of the control mice (G1), indicating that LC0033-1 and Ab0033 have different tumor inhibitory effects at different dosages. Moreover, LC0033-1 treatment showed dose-dependent tumor inhibition and exhibited a considerable tumor inhibitory effect at a low dosage (G5, TGI_{TV} %=80.3%). Ab0033 at a higher dosage (G4, TGI_{TV} %=84.1%) showed comparable anti-tumor activity to LC0033-1 at a medium dosage (G6, TGI_{TV} %=85.3%), but failed to inhibit tumor growth at a low (G2, TGI_{TV} %=40.0%) and medium dosage (G3, TGI_{TV} %=22.9%).

At the same dosage and dosing frequency, LC0033-1 (G5-G7) exhibited a better efficacy than Ab0033 (G2-G4), indicating that the in vivo tumor inhibitory effect of the anti-PD-L1 antibody was effectively improved upon fused with IL21.

As seen from FIG. 13, comparing to an equimolar of Ab0033 (G2), LC0033-1 at 3 mg/kg (G5) effectively inhibited tumor growth.

We also studied the data from individual mice within each treatment group. In the 3 mg/kg LC0033-1 group (G5), the growth of tumors in all the mice was slow, indicating inhibition of the tumor growth. While in the 2.73 mg/kg Ab0033 group (G2), only one mouse's tumor suffered significant inhibition.

For LC0033-1, the anti-tumor activity in the medium-dose group (G6) is higher than that of the low-dose group (G5); Ab0033 at corresponding dosages (G3 vs G2) did not exhibit significant anti-tumor activity. High-dose Ab0033 (G4) effectively inhibited tumor growth, with comparable anti-tumor activity to low-dose LC0033-1 (G5). The anti-tumor activity of LC0033-1 further increased at a high dosage (G7), but did not exhibit a significant increase in tumor inhibition when compared to the low-dose group (G5), i.e., no significant dose-dependent effect was observed, possibly due to that LC0033-1 at 3 mg/kg already exhibited a significant anti-tumor activity, and therefore, for the MC38 tumor model, further increase in the dosage of LC0033-1 did not completely eliminate the tumor.

Sequence Listing

| SEQ | | |
|---|---|---|
| ID No. | Description | Sequence |
| 1 | Light chain | DIQMTQSPSSLSASVGDRVTITCRASQDVSTAVAWYQQKPGKAPKLLIY |
| | of Ab0001/ | SASFLYSGVPSRFSGSGSGTDFTLTISSLQPEDFATYYCQQYLYHPATFG |
| | Ab0030 | QGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSFNRGEC |
| 2 | Heavy chain | EVQLVESGGGLVQPGGSLRLSCAASGFTFSDSWIHWVRQAPGKGLEW |
| | of Ab0001 | VAWISPYGGSTYYADSVKGRFTISADTSKNTAYLQMNSLRAEDTAVYY |
| | | CARRHWPGGFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHN |
| | | AKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHE |
| | | ALHNHYTQKSLSLSPGK |
| 3 | Light chain | DIVLTQSPDFQSVTPKEKVTITCRASQSISNNLHWYQQKPDESPKLLIKY |
| | of Ab0002/ | ASQSISGIPSRFSGSGSGTDFTLTINSVEAEDFAMYFCQQSKSWPFTFGQ |
| | Ab0033 | GTRLEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVT |
| | | HQGLSSPVTKSFNRGEC |
| 4 | Heavy chain | QVQLQESGPGLVKPSETLSLTCTVSGYSITSGYNWHWIRQFPGNGLEW |
| | of Ab0002 | MGYIHHSSITNYNPSLKSRITISRDTSKNQFSLKLSSVTAADTATYYCAR |
| | | EGYDYDWFAYWGQGTLVTVSSASTKGPSVFPLAPSSKSTSGGTAALGC |
| | | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAK |
| | | TKPREEQYASTYRVVSVLTVLHQDWLNGKEYKCKVSNKALPAPIEKTI |
| | | SKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEA |
| | | LHNHYTQKSLSLSPGK |
| 5 | Acceptor | GGGGSGGGGS |
| | substrate | |
| | recognition | |
| | sequence | |
| | and spacer | |
| 6 | Spacer and | GALPETGG |
| | donor | |
| | substrate | |
| | recognition | |
| | sequence | |
| 7 | hIL21 | MRSSPGNMERIVICLMVIFLGTLVHKSSSQGQDRHMIRMRQLIDIVDQL |
| | isoform 1 | KNYVNDLVPEFLPAPEDVETNCEWSAFSCFQKAQLKSANTGNNERIIN |
| | precursor | VSIKKLKRKPPSTNAGRRQKHRLTCPSCDSYEKKPPKEFLERFKSLLQK |
| | | MIHQHLSSRTHGSEDS |
| 8 | hIL21 | MRSSPGNMERIVICLMVIFLGTLVHKSSSQGQDRHMIRMRQLIDIVDQL |
| | isoform 2 | KNYVNDLVPEFLPAPEDVETNCEWSAFSCFQKAQLKSANTGNNERIIN |
| | precursor | VSIKKLKRKPPSTNAGRRQKHRLTCPSCDSYEKKPPKEFLERFKSLLQK |
| | | VSTLSFI |
| 9 | hIL21 | HKSSSQGQDRHMIRMRQLIDIVDQLKNYVNDLVPEFLPAPEDVETNCE |
| | isoform 1 | WSAFSCFQKAQLKSANTGNNERIINVSIKKLKRKPPSTNAGRRQKHRL |
| | | TCPSCDSYEKKPPKEFLERFKSLLQKMIHQHLSSRTHGSEDS |
| 10 | hIL21 | HKSSSQGQDRHMIRMRQLIDIVDQLKNYVNDLVPEFLPAPEDVETNCE |
| | isoform 2 | WSAFSCFQKAQLKSANTGNNERIINVSIKKLKRKPPSTNAGRRQKHRL |
| | | TCPSCDSYEKKPPKEFLERFKSLLQKVSTLSFI |
| 11 | Cytokine | HKSSSQGQDRHMIRMRQLIDIVDQLKNYVNDLVPEFLPAPEDVETNCE |
| | moiety | WSAFSCFQKAQLKSANTGNNERIINVSIKKLKRKPPSTNAGRRQKHRL |
| | derived from | TCPSCDSYEKKPPKEFLERFKSLLQKMIHQHLSSRTHGSEDSGALPETG |
| | hIL21 | G |
| | isoform 1 | |
| 12 | Cytokine | HKSSSQGQDRHMIRMRQLIDIVDQLKNYVNDLVPEFLPAPEDVETNCE |
| | moiety | WSAFSCFQKAQLKSANTGNNERIINVSIKKLKRKPPSTNAGRRQKHRL |
| | derived from | TCPSCDSYEKKPPKEFLERFKSLLQKVSTLSFIGALPETGG |
| | hIL21 | |
| | isoform 2 | |
| 13 | N-terminal | MRSSPGNMERIVICLMVIFLGTLV |
| | signal peptide | |
| | for hIL21 | |
| 14 | Heavy chain | GGGGSGGGGSEVQLVESGGGLVQPGGSLRLSCAASGFTFSDSWIHWV |
| | of Ab0030 | RQAPGKGLEWVAWISPYGGSTYYADSVKGRFTISADTSKNTAYLQMNS |
| | | LRAEDTAVYYCARRHWPGGFDYWGQGTLVTVSSASTKGPSVFPLAPS |
| | | SKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKENWY |
| | | VDGVEVHNAKTKPREEQYASTYRVVSVLTVLHQDWLNGKEYKCKVS |
| | | NKALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| 15 | Heavy chain | GGGGSGGGGSQVQLQESGPGLVKPSETLSLTCTVSGYSITSGYNWHWI |
| | of Ab0033 | RQFPGNGLEWMGYIHHSSITNYNPSLKSRITISRDTSKNQFSLKLSSVTA |
| | | ADTATYYCAREGYDYDWFAYWGQGTLVTVSSASTKGPSVFPLAPSSKS |
| | | TSGGTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSNK |
| | | ALPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVF |
| | | SCSVMHEALHNHYTQKSLSLSPGK |
| 16 | N-terminal | MDMRVPAQLLGLLLLWLRGARC |
| | light chain | |
| | signal | |
| | peptide | |
| 17 | N-terminal | MEFGLSWVFLVAILKGVQC |
| | heavy chain | |
| | signal | |
| | peptide | |
| 18 | Heavy chain | HKSSSQGQDRHMIRMRQLIDIVDQLKNYVNDLVPEFLPAPEDVETNCE |
| | moiety of | WSAFSCFQKAQLKSANTGNNERIINVSIKKLKRKPPSTNAGRRQKHRL |
| | LC0030-1 | TCPSCDSYEKKPPKEFLERFKSLLQKMIHQHLSSRTHGSEDSGALPETG |
| | and LC0030-2 | GGGSGGGGSEVQLVESGGGLVQPGGSLRLSCAASGFTFSDSWIHWVR |
| | | QAPGKGLEWVAWISPYGGSTYYADSVKGRFTISADTSKNTAYLQMNSL |
| | | RAEDTAVYYCARRHWPGGFDYWGQGTLVTVSSASTKGPSVFPLAPSS |
| | | KSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWY |
| | | VDGVEVHNAKTKPREEQYASTYRVVSVLTVLHQDWLNGKEYKCKVS |
| | | NKALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQGN |
| | | VFSCSVMHEALHNHYTQKSLSLSPGK |
| 19 | Heavy chain | HKSSSQGQDRHMIRMRQLIDIVDQLKNYVNDLVPEFLPAPEDVETNCE |
| | moiety of | WSAFSCFQKAQLKSANTGNNERIINVSIKKLKRKPPSTNAGRRQKHRL |
| | LC0033-1 | TCPSCDSYEKKPPKEFLERFKSLLQKMIHQHLSSRTHGSEDSGALPETG |
| | and LC0033-2 | GGGSGGGGSQVQLQESGPGLVKPSETLSLTCTVSGYSITSGYNWHWIR |
| | | QFPGNGLEWMGYIHHSSITNYNPSLKSRITISRDTSKNQFSLKLSSVTAA |
| | | DTATYYCAREGYDYDWFAYWGQGTLVTVSSASTKGPSVFPLAPSSKST |
| | | SGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVDG |
| | | VEVHNAKTKPREEQYASTYRVVSVLTVLHQDWLNGKEYKCKVSNKA |
| | | LPAPIEKTISKAKGQPREPQVYTLPPSREEMTKNQVSLTCLVKGFYPSDI |
| | | AVEWESNGQPENNYKTTPPVLDSDGSFFLYSKLTVDKSRWQQGNVFSC |
| | | SVMHEALHNHYTQKSLSLSPGK |
| 20 | Heavy chain | HKSSSQGQDRHMIRMRQLIDIVDQLKNYVNDLVPEFLPAPEDVETNCE |
| | moiety of | WSAFSCFQKAQLKSANTGNNERIINVSIKKLKRKPPSTNAGRRQKHRL |
| | LC0030′-1 | TCPSCDSYEKKPPKEFLERFKSLLQKVSTLSFIGALPETGGGGSGGGGS |
| | and | EVQLVESGGGLVQPGGSLRLSCAASGFTFSDSWIHWVRQAPGKGLEW |
| | LC0030′-2 | VAWISPYGGSTYYADSVKGRFTISADTSKNTAYLQMNSLRAEDTAVYY |
| | | CARRHWPGGFDYWGQGTLVTVSSASTKGPSVFPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHN |
| | | AKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHE |
| | | ALHNHYTQKSLSLSPGK |
| 21 | Heavy chain | HKSSSQGQDRHMIRMRQLIDIVDQLKNYVNDLVPEFLPAPEDVETNCE |
| | moiety of | WSAFSCFQKAQLKSANTGNNERIINVSIKKLKRKPPSTNAGRRQKHRL |
| | LC0033′-1 | TCPSCDSYEKKPPKEFLERFKSLLQKVSTLSFIGALPETGGGGSGGGGS |
| | and | QVQLQESGPGLVKPSETLSLTCTVSGYSITSGYNWHWIRQFPGNGLEW |
| | LC0033′-2 | MGYIHHSSITNYNPSLKSRITISRDTSKNQFSLKLSSVTAADTATYYCAR |
| | | EGYDYDWFAYWGQGTLVTVSSASTKGPSVFPLAPSSKSTSGGTAALGC |
| | | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAK |
| | | TKPREEQYASTYRVVSVLTVLHQDWLNGKEYKCKVSNKALPAPIEKTI |
| | | SKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEA |
| | | LHNHYTQKSLSLSPGK |
| 22 | CDRL1 of | RASQDVSTAVA |
| | Ab0001 | |
| 23 | CDRL2 of | SASFLYS |
| | Ab0001 | |
| 24 | CDRL3 of | QQYLYHPAT |
| | Ab0001 | |
| 25 | CDRH1 of | DSWIH |
| | Ab0001 | |
| 26 | CDRH2 of | WISPYGGSTYYADSVKG |
| | Ab0001 | |
| 27 | CDRH3 of | RHWPGGFDY |
| | Ab0001 | |
| 28 | CDRL1 of | RASQSISNNLH |
| | Ab0002 | |
| 29 | CDRL2 of | YASQSIS |
| | Ab0002 | |
| 30 | CDRL3 of | QQSKSWPFT |
| | Ab0002 | |
| 31 | CDRH1 of | SGYNWH |
| | Ab0002 | |
| 32 | CDRH2 of | YIHHSSITNYNPSLKS |
| | Ab0002 | |
| 33 | CDRH3 of | EGYDYDWFAY |
| | Ab0002 | |
| 34 | VL of | DIQMTQSPSSLSASVGDRVTITCRASQDVSTAVAWYQQKPGKAPKLLIY |
| | Ab0001 | SASFLYSGVPSRFSGSGSGTDFTLTISSLQPEDFATYYCQQYLYHPATFG |
| | | QGTKVEIK |
| 35 | VH of | EVQLVESGGGLVQPGGSLRLSCAASGFTFSDSWIHWVRQAPGKGLEW |
| | Ab0001 | VAWISPYGGSTYYADSVKGRFTISADTSKNTAYLQMNSLRAEDTAVYY |
| | | CARRHWPGGFDYWGQGTLVTVSS |
| 36 | VL of | DIVLTQSPDFQSVTPKEKVTITCRASQSISNNLHWYQQKPDESPKLLIKY |
| | Ab0002 | ASQSISGIPSRFSGSGSGTDFTLTINSVEAEDFAMYFCQQSKSWPFTFGQ |
| | | GTRLEIK |
| 37 | VH of | QVQLQESGPGLVKPSETLSLTCTVSGYSITSGYNWHWIRQFPGNGLEW |
| | Ab0002 | MGYIHHSSITNYNPSLKSRITISRDTSKNQFSLKLSSVTAADTATYYCAR |
| | | EGYDYDWFAYWGQGTLVTVSS |
| SEQ | | |
| ID No. | Sequence | Description |
| 38 | LPXTG | X is any natural or unnatural amino acid |
| 39 | LPXTGG | X is any natural or unnatural amino acid |
| 40 | NPQTN | |
| 41 | NPKTG | |
| 42 | LAXTG | X is any natural or unnatural amino acid |
| 43 | LPQTSEQ | |
| 44 | LPETG | |
| 45 | LPETGG | |
| 46 | NPXTGG | X is any natural or unnatural amino acid |
| 47 | LPXTA | X is any natural or unnatural amino acid |
| 48 | LAXTGG | X is any natural or unnatural amino acid |
| 49 | LPXTG | X is selected from the group consisting of A, R, E, N, D, Q, |
| | | I, L and K |
| 50 | LPXTGX | X at position 3 is any single amino acid that is natural or |
| | | unnatural; X at position 6 is absent or an amino acid sequence |
| | | comprising 1-10 amino acids that can be independently any |
| | | natural or unnatural amino acid |
| 51 | LPXTX | X at position 3 can be any natural or unnatural amino acid; |
| | | X at position 5 is 2-20 glycines |
| 52 | NPXTX | X at position 3 can be any natural or unnatural amino acid; |
| | | X at position 5 is 2-20 glycines |
| 53 | LAXTX | X at position 3 can be any natural or unnatural amino acid; |
| | | X at position 5 is 2-20 glycines |
| 54 | LPETGGG | |
| 55 | LPQTGGG | |
| 56 | NPQTX | X is 2-20 glycines |
| 57 | NPKTX | X is 2-20 glycines |
| 58 | GSGGGGS | |
| 59 | GXSX | X at position 2 is absent or 1-4 glycines; X at position 4 is |
| | | ((G)ₛS)ₜ in which s is an integer or 1-10 and t is 0 or an |
| | | integer of 1-10 |
| 60 | GALPETGGGGSGGGGS | |

1. A fusion protein having the structure of formula (I) or (I′):
[B-Sp₁-Lk-Sp₂]ₘ-A (I)
A-[Sp₁-Lk-Sp₂-B]ₘ (I′)
wherein
A is an anti-PD-L1 antibody;
B is an interleukin 21 (IL21);
Sp₁ and Sp2 are each independently a spacer or absent,
Lk is a linker formed by conjugation of an acceptor substrate recognition sequence and a donor substrate recognition sequence of a Sortase;
m is an integer of 1-4.

2. The fusion protein according to claim 1, wherein the acceptor substrate recognition sequence comprises 1-100 series-connected structure units which are selected from the group consisting of glycine (G) and alanine (A), optionally wherein the acceptor substrate recognition sequence comprises the sequence of (G)ₙ, wherein n is an integer of 2-20, especially 3.

3. (canceled)

4. The fusion protein according to claim 1, wherein the donor substrate recognition sequence comprises the sequence of X₁X₂X₃TX₄X₅, wherein, X₁ is leucine (L) or asparagine (N), X₂ is proline (P) or alanine (A), X₃ is any natural or unnatural amino acid, X₄ is glycine (G), serine (S) or asparagine (N) or absent, X₅ is an amino acid sequence comprising 1-10 amino acids or absent.

5. The fusion protein according to claim 4, wherein X₁X₂X₃TX₄X₅ is LPX₃TG, LPX₃TGG, NPQTN, NPKTG, LAX₃TG or LPQTSEQ, wherein X₃ is any natural or unnatural amino acid, optionally wherein X₁X₂X₃TX₄X₅ is LPETG or LPETGG.

6. (canceled)

7. The fusion protein according to claim 1, wherein the spacer comprises 1-100 series-connected structure units which are selected from the group consisting of glycine (G), alanine (A) and serine (S);
preferably, the spacer is selected from the group consisting of GA, GGG, GAG and (G)ₒS((G)ₛS)ₜ, wherein o is an integer of 1-5; s is an integer of 1-10, especially 4; t is 0 or an integer of 1-10.

8. The fusion protein according to claim 1, wherein Lk is X1X2X3T(G)ₙ, the fusion protein has the structure of (II) or (II′):
[B-Sp₁-X₁X₂X₃T(G)ₙ-Sp₂]ₘ-A (II)
A-[Sp₁-X₁X₂X₃T(G)ₙ-Sp₂-B]ₘ (II′)
wherein, X₁ is leucine (L) or asparagine (N), X₂ is proline (P) or alanine (A), X₃ is any natural or unnatural amino acid, n is an integer of 2-20,
m is 1 or 2;
A, B, Sp₁ and Sp₂ are as defined in claim 1.

9. The fusion protein according to claim 8, wherein X₁X₂X₃T(G)ₙ is LPX₃T(G)ₙ, NPX₃T(G)ₙ or LAX₃T(G)ₙ, X₃ is any natural or unnatural amino acid, n is an integer of 2-20.

10. The fusion protein according to claim 9, wherein the fusion protein has the structure of (III) or (III′):
[B-GALPETGGGGSGGGGS]ₘ-A (III)
A-[GALPETGGGGSGGGGS-B]ₘ (III′)

11. The fusion protein according to claim 1, wherein the anti-PD-L1 antibody is Atezolizumab, Avelumab, Durvalumab, 3F2 or an engineered antibody therefrom;
preferably, the anti-PD-L1 antibody comprises a light chain variable region (VL) and a heavy chain variable region (VH), wherein
(1) the VL comprises a CDRL1 sequence as shown in SEQ ID NO:22, a CDRL2 sequence as shown in SEQ ID NO:23 and a CDRL3 sequence as shown in SEQ ID NO:24, and the VH comprises a CDRH1 sequence as shown in SEQ ID NO:25, a CDRH2 sequence as shown in SEQ ID NO:26 and a CDRH3 sequence as shown in SEQ ID NO:27; or
(2) the VL comprises a CDRL1 sequence as shown in SEQ ID NO:28, a CDRL2 sequence as shown in SEQ ID NO:29 and a CDRL3 sequence as shown in SEQ ID NO:30, the VH comprises a CDRH1 sequence as shown in SEQ ID NO:31, a CDRH2 sequence as shown in SEQ ID NO:32 and a CDRH3 sequence as shown in SEQ ID NO:33;
more preferably, the VL comprises the amino acid sequence of SEQ ID NO:34, and the VH comprises the amino acid sequence of SEQ ID NO:35; or the VL comprises the amino acid sequence of SEQ ID NO:36, and the VH comprises the amino acid sequence of SEQ ID NO:37;
most preferably, the anti-PD-L1 antibody comprises a light chain and a heavy chain, wherein
(1) the light chain comprises the amino acid sequence of SEQ ID NO:1 and the heavy chain comprises the amino acid sequence of SEQ ID NO:2; or
(2) the light chain comprises the amino acid sequence of SEQ ID NO:3 and the heavy chain comprises the amino acid sequence of SEQ ID NO:4.

12. The fusion protein according to claim 11, wherein the IL21 is a wild-type IL21 or a variant thereof;
preferably, the IL21 comprises the amino acid sequence of SEQ ID NO:9 or SEQ ID NO:10.

13. The fusion protein according to claim 1, wherein the fusion protein has the structure of formula (III):
[B-GALPETGGGGSGGGGS]ₘ-A (III)
wherein
A is an anti-PD-L1 antibody comprising a light chain having the amino acid sequence of SEQ ID NO:1 and a heavy chain having the amino acid sequence of SEQ ID NO:2, or a light chain having the amino acid sequence of SEQ ID NO:3 and a heavy chain having the amino acid sequence of SEQ ID NO:4;
B is an IL21 comprising the amino acid sequence of SEQ ID NO:9 or SEQ ID NO:10;
m is 1 or 2.

14. The fusion protein according to claim 11, wherein the IL21 is conjugated at the N-terminus of the light chain and/or heavy chain of the anti-PD-L1 antibody;
preferably, the IL21 is conjugated at the N-terminus of the heavy chain of the anti-PD-L1 antibody.

15. The fusion protein according to claim 1, wherein the fusion protein comprises:
(a) two identical heavy chain moieties and two identical light chain moieties, and wherein
(1) each of the heavy chain moieties comprises the amino acid sequence of SEQ ID NO:18 or SEQ ID NO:20; and each of the light chain moieties comprises the amino acid sequence of SEQ ID NO:1; or
(2) each of the heavy chain moieties comprises the amino acid sequence of SEQ ID NO:19 or SEQ ID NO:21; and each of the light chain moieties comprises the amino acid sequence of SEQ ID NO:3;
or
(b) two different heavy chain moieties and two identical light chain moieties, and wherein
(1) one of the heavy chain moieties comprises the amino acid sequence of SEQ ID NO:18 or SEQ ID NO:20, the other one of the heavy chain moieties comprises the amino acid sequence of SEQ ID NO:14; and each of the light chain moieties comprises the amino acid sequence of SEQ ID NO:1; or
(2) one of the heavy chain moieties comprises the amino acid sequence of SEQ ID NO:19 or SEQ ID NO:21, the other one of the heavy chain moieties comprises the amino acid sequence of SEQ ID NO:15; and each of the light chain moieties comprises the amino acid sequence of SEQ ID NO:3.

16. A method of treating a disease in a subject in need thereof, comprising administering the fusion protein according to claim 1 to the subject;
preferably, the disease is a cancer;
more preferably, the cancer is selected from melanoma, ovarian cancer, breast cancer, Merkel cell carcinoma, lung cancer, renal cell cancer, bladder cancer, colon cancer, colon adenocarcinoma, colorectal cancer, head and neck squamous cell carcinoma, mesothelioma, virally induced cancers, cervical cancer, nasopharyngeal cancer, soft tissue sarcomas, hematological malignancies, gastric cancer, gastric or gastroesophageal junction (GEJ) adenocarcinoma, esophagus cancer, squamous cell carcinoma of the esophagus, endometrial carcinoma, primary mediastinal B-cell lymphoma (PMBCL), urothelial carcinoma, microsatellite instability-high (MSI-H) or a mismatch repair deficient (dMMR) solid tumor, hepatocellular carcinoma, lymphoma, Hodgkin lymphoma, non-Hodgkin's lymphoma, diffuse large B-cell lymphoma, renal cell carcinoma (RCC), cutaneous squamous cell carcinoma (cSCC), triple-negative breast cancer (TNBC), non-small cell lung cancer and bladder cancer.

17. A method for producing the fusion protein according to claim 1, comprising the following steps:
(a-1) providing an antibody moiety having the structure of formula (I-1) and a cytokine moiety having the structure of formula (I-2)
[Lkₐ-Sp₂]ₘ-A (I-1)
B-Sp₁-Lk_{d} (I-2);
and
(b-1) conjugating the antibody moiety with the cytokine moiety in the presence of a Sortase to obtain the fusion protein having the structure of formula (I)
B-Sp₁-Lk_{d} (I-2);
or
(a-2) providing an antibody moiety having the structure of formula (I′-1) and a cytokine moiety having the structure of formula (I′-2)
A-[Sp₁-Lk_{d}]ₘ (I′-1)
Lkₐ-Sp₂-B (I′-2);
and
(b-2) conjugating the antibody moiety with the cytokine moiety in the presence of a Sortase to obtain the fusion protein having the structure of formula (I′)
A-[Sp₁-Lk-Sp₂-B]ₘ (I′);
wherein
Lkₐ is an acceptor substrate recognition sequence of the Sortase, Lk_{d} is a donor substrate recognition sequence of the Sortase;
A, B, Sp₁, Sp₂, Lk and m are as defined in claim 1.

18. The method according to claim 17, wherein step (a-1) further comprising:
(a-1-1) adding Lkₐ-Sp₂ to the N-terminus of the heavy chain of the anti-PD-L1 antibody to obtain the antibody moiety having the structure of formula (I-1)
[Lkₐ-Sp₂]ₘ-A (I-1);
and
(a-1-2) adding Sp₁-Lk_{d} to the C-terminus of an IL21 to obtain the cytokine moiety having the structure of formula (I-2)
B-Sp₁-Lk_{d} (I-2).

19. The method according to claim 17, wherein step (a-2) further comprising:
(a-2-1) adding Sp₁-Lk_{d} to the C-terminus of the heavy chain of the anti-PD-L1 antibody to obtain the antibody moiety having the structure of formula (I′-1)
A-[Sp₁-Lk_{d}]ₘ (I′-1)
and
(a-2-2) adding Lkₐ-Sp₂ to the N-terminus of an IL21 to obtain the cytokine moiety having the structure of formula (I′-2)
Lkₐ-Sp₂-B (I′-2).

20. The method according to claim 17, wherein the acceptor substrate recognition sequence comprises 1-100 series-connected structure units which are selected from the group consisting of glycine (G) and alanine (A), optionally wherein the acceptor substrate recognition sequence comprises the sequence of (G)ₙ, wherein n is an integer of 2-20, especially 3;
the donor substrate recognition sequence comprises the sequence of X₁X₂X₃TX₄X₅, wherein, X₁ is leucine (L) or asparagine (N), X₂ is proline (P) or alanine (A), X₃ is any natural or unnatural amino acid, X₄ is glycine (G), serine (S) or asparagine (N) or absent, X₅ is an amino acid sequence comprising 1-10 amino acids or absent, optionally wherein X₁X₂X₃TX₄X₅ is LPX₃TG, LPX₃TGG, NPQTN, NPKTG, LAX₃TG or LPQTSEQ, wherein X₃ is any natural or unnatural amino acid, further optionally wherein X₁X₂X₃TX₄X₅ is LPETG or LPETGG; and
the spacer comprises 1-100 series-connected structure units which are selected from the group consisting of glycine (G), alanine (A) and serine (S), preferably, the spacer is selected from the group consisting of GA, GGG, GAG and (G)ₒS((G)ₛS)ₜ, wherein o is an integer of 1-5; s is an integer of 1-10, especially 4; t is 0 or an integer of 1-10.

21. (canceled)

22. (canceled)

23. The method according to claim 17, wherein the anti-PD-L1 antibody is Atezolizumab, Avelumab, Durvalumab, 3F2 or an engineered antibody therefrom;
preferably, the anti-PD-L1 antibody comprises a light chain variable region (VL) and a heavy chain variable region (VH), wherein
(1) the VL comprises a CDRL1 sequence as shown in SEQ ID NO:22, a CDRL2 sequence as shown in SEQ ID NO:23 and a CDRL3 sequence as shown in SEQ ID NO:24, and the VH comprises a CDRH1 sequence as shown in SEQ ID NO:25, a CDRH2 sequence as shown in SEQ ID NO:26 and a CDRH3 sequence as shown in SEQ ID NO:27; or
(2) the VL comprises a CDRL1 sequence as shown in SEQ ID NO:28, a CDRL2 sequence as shown in SEQ ID NO:29 and a CDRL3 sequence as shown in SEQ ID NO:30, the VH comprises a CDRH1 sequence as shown in SEQ ID NO:31, a CDRH2 sequence as shown in SEQ ID NO:32 and a CDRH3 sequence as shown in SEQ ID NO:33;
more preferably, the VL comprises the amino acid sequence of SEQ ID NO:34, and the VH comprises the amino acid sequence of SEQ ID NO:35; or the VL comprises the amino acid sequence of SEQ ID NO:36, and the VH comprises the amino acid sequence of SEQ ID NO:37;
most preferably, the anti-PD-L1 antibody comprises a light chain and a heavy chain, wherein
(1) the light chain comprises the amino acid sequence of SEQ ID NO:1 and the heavy chain comprises the amino acid sequence of SEQ ID NO:2; or
(2) the light chain comprises the amino acid sequence of SEQ ID NO:3 and the heavy chain comprises the amino acid sequence of SEQ ID NO:4.

24. The method according to claim 17, wherein the IL21 is a wild-type IL21 or a variant thereof;
preferably, the IL21 comprises the amino acid sequence of SEQ ID NO:9 or SEQ ID NO:10.


SEQUENCE LISTING (37 sequences)

SEQ ID NO: 1 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
DIVLTQSPDFQSVTPKEKVTITCRASQSISNNLHWYQQKPDESPKLLIKYASQSISGIPSRFSGSGSGTDFTLTINSVEAEDFAMYFCQQSKSWPFTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, synthetic construct
QVQLQESGPGLVKPSETLSLTCTVSGYSITSGYNWHWIRQFPGNGLEWMGYIHHSSITNYNPSLKSRITISRDTSKNQFSLKLSSVTAADTATYYCAREGYDYDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 10 aa, synthetic construct
GGGGSGGGGS

SEQ ID NO: 6 - protein, 8 aa, synthetic construct
GALPETGG

SEQ ID NO: 7 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 8 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 9 - protein, 138 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 10 - protein, 129 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 11 - protein, 146 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGALPETGG

SEQ ID NO: 12 - protein, 137 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIGALPETGG

SEQ ID NO: 13 - protein, 24 aa, synthetic construct
MRSSPGNMERIVICLMVIFLGTLV

SEQ ID NO: 14 - protein, 458 aa, synthetic construct
GGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 459 aa, synthetic construct
GGGGSGGGGSQVQLQESGPGLVKPSETLSLTCTVSGYSITSGYNWHWIRQFPGNGLEWMGYIHHSSITNYNPSLKSRITISRDTSKNQFSLKLSSVTAADTATYYCAREGYDYDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 22 aa, synthetic construct
MDMRVPAQLLGLLLLWLRGARC

SEQ ID NO: 17 - protein, 19 aa, synthetic construct
MEFGLSWVFLVAILKGVQC

SEQ ID NO: 18 - protein, 602 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGALPETGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 603 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGALPETGGGGSGGGGSQVQLQESGPGLVKPSETLSLTCTVSGYSITSGYNWHWIRQFPGNGLEWMGYIHHSSITNYNPSLKSRITISRDTSKNQFSLKLSSVTAADTATYYCAREGYDYDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 593 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIGALPETGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 594 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIGALPETGGGGSGGGGSQVQLQESGPGLVKPSETLSLTCTVSGYSITSGYNWHWIRQFPGNGLEWMGYIHHSSITNYNPSLKSRITISRDTSKNQFSLKLSSVTAADTATYYCAREGYDYDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 11 aa, synthetic construct
RASQDVSTAVA

SEQ ID NO: 23 - protein, 7 aa, synthetic construct
SASFLYS

SEQ ID NO: 24 - protein, 9 aa, synthetic construct
QQYLYHPAT

SEQ ID NO: 25 - protein, 5 aa, synthetic construct
DSWIH

SEQ ID NO: 26 - protein, 17 aa, synthetic construct
WISPYGGSTYYADSVKG

SEQ ID NO: 27 - protein, 9 aa, synthetic construct
RHWPGGFDY

SEQ ID NO: 28 - protein, 11 aa, synthetic construct
RASQSISNNLH

SEQ ID NO: 29 - protein, 7 aa, synthetic construct
YASQSIS

SEQ ID NO: 30 - protein, 9 aa, synthetic construct
QQSKSWPFT

SEQ ID NO: 31 - protein, 6 aa, synthetic construct
SGYNWH

SEQ ID NO: 32 - protein, 16 aa, synthetic construct
YIHHSSITNYNPSLKS

SEQ ID NO: 33 - protein, 10 aa, synthetic construct
EGYDYDWFAY

SEQ ID NO: 34 - protein, 107 aa, synthetic construct [sequence omitted]

SEQ ID NO: 35 - protein, 118 aa, synthetic construct [sequence omitted]

SEQ ID NO: 36 - protein, 107 aa, synthetic construct
DIVLTQSPDFQSVTPKEKVTITCRASQSISNNLHWYQQKPDESPKLLIKYASQSISGIPSRFSGSGSGTDFTLTINSVEAEDFAMYFCQQSKSWPFTFGQGTRLEIK

SEQ ID NO: 37 - protein, 119 aa, synthetic construct
QVQLQESGPGLVKPSETLSLTCTVSGYSITSGYNWHWIRQFPGNGLEWMGYIHHSSITNYNPSLKSRITISRDTSKNQFSLKLSSVTAADTATYYCAREGYDYDWFAYWGQGTLVTVSS